(19) United States
(12) Patent Application Publication
(10) Pub. No.: US 2025/0050038 A1
(43) Pub. Date: Feb. 13, 2025

(54) ADAPTABLE REUSEABLE ACCESSORY, DETECTING REVERSIBLE ATTACHMENT OF MEDICATION DELIVERY DEVICE THEREIN, AND KIT THEREOF

(71) Applicant: Bigfoot Biomedical, Inc., Milpitas, CA (US)

(72) Inventors: Christopher Kovalchick, Milpitas, CA (US); Jorge A. Balderrama Canales, Milpitas, CA (US); Matthew Clemente, Milpitas, CA (US); Rachael Price, Milpitas, CA (US); Alec Lindeman, Santa Clara, CA (US); Peter A. Holst, Santa Clara, CA (US); Jane Zhang, Milpitas, CA (US)

(21) Appl. No.: 18/798,485

(22) Filed: Aug. 8, 2024

Related U.S. Application Data
(60) Provisional application No. 63/518,203, filed on Aug. 8, 2023.

(51) Int. Cl. A61M 5/50; A61M 5/32

(52) U.S. Cl. CPC A61M 5/5086; A61M 5/3202; A61M 2205/14; A61M 2205/18

(57) ABSTRACT

A reusable accessory for a medication delivery device includes a housing, an adaptive element coupled to the housing and configured to reversibly attach to a medication delivery device received in the housing, and one or more attachment sensors configured to detect the adaptive element reversibly attaching to the medication delivery device. A method for detecting a medication delivery device in a reusable accessory includes determining, based on sensing data from one or more attachment sensors, whether the medication delivery device is reversibly attached, via an adaptable element, to a housing of the reusable accessory, and presenting, via at least one user interface, a status of the medication delivery device. A reusable accessory adaption kit comprises a first reusable accessory, a first adaptive element configured for reversibly attaching a first medication delivery device, and a second adaptive element configured for reversibly attaching a second medication delivery device.

### TECHNICAL FIELD

This disclosure generally relates to receiving of medication delivery devices in reusable accessories. More specifically, this disclosure relates to detection of reversable attachment of medication delivery devices in reusable accessories.

### BACKGROUND

Medication delivery devices are used to deliver a medication to a person. Some medication delivery devices can be used by a person to self-administer medication. Medications delivered by medication delivery devices are not particularly limited to a specific type of external biologically effective drug. For example, medications delivered by medication delivery devices can include, but are not limited to, diabetes medications (e.g., insulin, analogs of insulin, glucagon), blood thinners (e.g., heparin), palliative care pain medications, growth hormone deficiency treatment medications (e.g., somatotropin), fertility treatments (e.g., human chorionic gonadotropin), follicle stimulating hormones, etc. Reuseable accessories are made to be engaged with a medication delivery device when not in use.

### BRIEF SUMMARY

The various embodiments described below provide benefits and/or solve one or more of the foregoing or other problems in the art with devices and methods for a reuseable accessory for medication delivery devices. Embodiments include a reusable accessory for use with medication delivery devices, in which the medication delivery device includes one or more adaptive elements and one or more attachment sensors. The adaptive element(s) are configured to removably couple the reusable accessory to a medication delivery device, while the attachment sensor(s) can ensure a proper connection between the reusable accessory and the mediation delivery device delivery device.

In an embodiment, a reusable accessory for receiving a medication delivery device includes a housing, an adaptive element coupled to the housing, and one or more attachment sensors coupled to the housing. The housing is configured to receive a medication delivery device. The adaptive element is configured to reversibly attach to the medication delivery device. The one or more attachment sensors is configured to detect the adaptive element reversibly attaching to the medication delivery device.

In an embodiment, a method is directed to detecting a medication delivery device in a reusable accessory. The reusable accessory includes a housing configured to receive the medication delivery device, an adaptable element coupled to the housing, and one or more attachment sensors. The method includes receiving, via the one or more attachment sensors, sensing data for receiving the medication delivery device in the housing of the reusable accessory, and determining, based on the received sensing data, whether the medication delivery device is reversibly attached, via the adaptable element, to the housing of the reusable accessory. The method also includes presenting, via the at least one user interface, a status of the medication delivery device being reversibly attached to the reusable accessory via the adaptable element.

In an embodiment, a reusable accessory adaption kit includes a first reuseable accessory, a first adaptive element, and a second adaptive element different from the first adaptive element. The first reuseable accessory includes a housing and one or more attachment sensors coupled to the housing. The first adaptive element is configured to reversibly attach to a first medication delivery device while reversibly coupled to the housing of the reusable accessory. The second adaptive element is configured to reversibly attach to a second medication delivery device while reversibly coupled to the housing of the reusable accessory. The first medication delivery device has a geometry that is different from a geometry of the second medication delivery device. The one or more attachment sensors are configured to detect the reversible attachment of the first adaptive element to the first medication delivery device, when the first medication delivery device is received in the housing of the reuseable accessory. The one or more attachment sensors are also configured to detect the reversible attachment of the second adaptive element to the second medication delivery device, when the second medication delivery device is received in the housing of the reusable accessory.

In an embodiment, an adaptable reusable accessory kit includes a reusable accessory and a plurality of adaptive elements for a plurality of delivery medication devices with different geometries. The reuseable accessory includes a housing and one or more attachment sensors coupled to the housing. The plurality of adaptive elements are each configured to reversibly attach to a corresponding medication delivery device in the plurality of delivery medication devices while reversibly coupled to the housing of the reusable accessory. The one or more attachment sensors of the reuseable accessories are configured to detect, for each adaptive element in the plurality of adaptive elements when reversibly coupled to the housing, the reversible attaching of said adaptive element to the corresponding medication delivery device received in the housing.

### BRIEF DESCRIPTION OF THE DRAWINGS

The accompanying drawings illustrate various embodiments of systems, methods, and embodiments, and various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Non-limiting and non-exhaustive descriptions are described with reference to the following drawings. The components in the figures are not necessarily to scale, emphasis instead being placed upon illustrating principles. In the detailed description that follows, embodiments are described as illustrations only since various changes and modifications may become apparent to those skilled in the art from the following detailed description.

FIG. 1A illustrates an example medication delivery management system, according to an embodiment.

FIG. 1B illustrates a perspective view of a medication delivery device and a reuseable accessory, with the reuseable accessory separated from the medication delivery device, according to an embodiment.

FIG. 2 illustrates a block flow diagram of a method for detecting a reversible attachment of medication delivery device and a reuseable accessory, according to an embodiment.

FIG. 3 illustrates a side view of a reuseable accessory for a medication delivery device, according to an embodiment.

FIG. 4A illustrates a block flow chart of a method for detecting an insertion of a medication delivery device into a reuseable accessory, according to an embodiment.

FIG. 4B illustrates a block flow chart of a method for detecting a removal of a medication delivery device from a reuseable accessory, according to an embodiment.

FIG. 5 illustrates a side view of medication delivery devices with varying geometries, according to an embodiment.

FIG. 6 illustrates a side view of a reuseable accessory with an interchangeable collar separated from the reuseable accessory, according to an embodiment.

FIG. 7A illustrates a side perspective view of reuseable accessory and an interchangeable ring, with the interchangeable ring separated from the reuseable accessory, according to an embodiment.

FIG. 7B illustrates a side view of reuseable accessory and the interchangeable ring of FIG. 7A, with the ring reversibly coupled to the reuseable accessory, according to an embodiment.

FIG. 8 illustrates a top perspective view of a kit including pen caps and adaptive elements, according to an embodiment.

FIG. 9 is a side sectional view of a reuseable accessory having a clamping mechanism for medication delivery devices, according to an embodiment.

FIG. 10 is an exploded view of the clamping mechanism of the reuseable accessory in FIG. 9, according to an embodiment.

FIG. 11A-1 is a side view of the clamping mechanism of the reuseable accessory in FIG. 9, with the clamping mechanism in a first state, according to an embodiment. FIG. 11A-2 is a schematic view of the clamping mechanism in FIG. 11A-1, according to an embodiment.

FIG. 11B-1 is a side view of the clamping mechanism of the reuseable accessory in FIG. 9, with the clamping mechanism in a second state, according to an embodiment. FIG. 11B-2 is a schematic view of the clamping mechanism in FIG. 11B-1, according to an embodiment.

FIG. 11C-1 is a side view of the clamping mechanism of the reuseable accessory in FIG. 9, with the clamping mechanism in a third state, according to an embodiment. FIG. 11C-2 is a schematic view of the clamping mechanism in FIG. 11C-1, according to an embodiment.

FIG. 11D-1 is a side view of the clamping mechanism of the reuseable accessory in FIG. 9, with the clamping mechanism in a fourth state, according to an embodiment. FIG. 11D-2 is a schematic view of the clamping mechanism in FIG. 11D-1, according to an embodiment.

FIG. 11E-1 is a side view of the clamping mechanism of the reuseable accessory in FIG. 9, with the clamping mechanism in a fifth state, according to an embodiment. FIG. 11E-2 is a schematic view of the clamping mechanism in FIG. 11E-1, according to an embodiment.

FIG. 11F-1 is a side view of the clamping mechanism of the reuseable accessory in FIG. 9, with the clamping mechanism in a fifth state, according to an embodiment. FIG. 11F-2 is a schematic view of the clamping mechanism in FIG. 11F-1, according to an embodiment.

FIG. 12 is a partial perspective side view of a clicker portion, a clicker sensor, a circuit board, and a clicker retainer of the reusable accessory in FIG. 9, according to an embodiment.

Like reference characters represent like features.

### DETAILED DESCRIPTION

In the following detailed description, particular embodiments of the present disclosure are described herein with reference to the accompanying drawings, which form a part of the description. In this description, as well as in the drawings, like-referenced numbers represent elements that may perform the same, similar, or equivalent functions, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not intended to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Additionally, the present disclosure may be described herein in terms of functional block components and various processing steps of a method. It should be appreciated that such functional blocks may be realized by any number of hardware and/or software components configured to perform the specified functions.

The scope of the disclosure should be determined by the appended claims and their legal equivalents, rather than by the examples given herein. For example, the steps recited in any method claims may be executed in any order and are not limited to the order presented in the claims. Moreover, no element is essential to the practice of the disclosure unless specifically described herein as “critical” or “essential”.

This disclosure generally relates to reuseable accessories configured to receive medication delivery devices. More specifically, this disclosure generally relates to detecting receiving of medication delivery devices in reusable accessories. Medication delivery devices, systems thereof, and methods described herein may be used for and/or in conjunction with delivering various medications and managing various diseases in a patient. For example, medications delivered by medication delivery devices can include, but are not limited to, diabetes medications (e.g., insulin, analogs of insulin, glucagon), blood thinners (e.g., heparin), palliative care pain medications, growth hormone deficiency treatment medications (e.g., somatotropin), fertility treatments (e.g., human chorionic gonadotropin), follicle stimulating hormones, etc. Diabetes mellitus is a chronic metabolic disorder caused by the inability of a person's pancreas to produce sufficient amounts of the hormone insulin such that the person's metabolism is unable to provide for the proper absorption of sugar and starch. The inability to absorb those carbohydrates sometimes leads to hyperglycemia, i.e., the presence of an excessive amount of glucose within the blood plasma. Hyperglycemia has been associated with a variety of serious symptoms and life threatening long-term complications such as dehydration, ketoacidosis, diabetic coma, cardiovascular diseases, chronic renal failure, retinal damage, and nerve damages with the risk of amputation of extremities.

Often, a permanent therapy is necessary to maintain a proper blood glucose (i.e., amount of glucose in a person's bloodstream or a glucose value (also referred to herein as an “estimated glucose value”) representative of the same, such as a blood glucose level taken by a blood glucose meter or a glucose level generated by a glucose monitor) within normal limits. Maintaining a proper glucose level is conventionally achieved by regularly supplying insulin to a person with diabetes (PWD). Maintaining a proper glucose value may create a significant cognitive burden for a PWD (or a caregiver) and affect many aspects of the PWD's life. For example, the cognitive burden on a PWD may be attributed to, among other things, tracking meals and constant check-ins and minor course corrections of glucose values. The adjustments of glucose values by a PWD may include taking insulin, tracking insulin dosing and glucose, deciding how much insulin to take, how often to take it, where to inject the insulin, and how to time insulin doses in relation to meals and/or glucose fluctuations. A medication delivery device, when used to deliver insulin, can be referred to as an insulin pen. A reusable accessory, when used with an insulin pen, can be referred to as a cap or pen cap.

Embodiments reusable accessories, systems thereof, and methods thereof for medication delivery devices. Such medication delivery devices can be employed to provide multiple, daily delivery of medication, for example, a mixture of rapid-acting and long-acting insulin to a user. For example, medication delivery devices, systems, methods, disclosed herein may allow a user (e.g., a person with diabetes or PWD) to keep track of when a dose of medication was last delivered and monitor whether a dose was missed, with medication delivery devices with varying geometries.

In one or more examples, the disclosed devices, systems, and methods include a housing configured to receive a medication delivery device, one or more adaptive elements that reversibly attach to the medication delivery device, and one or more attachment sensors that detects the reversible attachment between the adaptive element(s) and the medication delivery device. In some examples, the disclosed devices, systems, and methods further include at least one user interface configured to communicate whether the medication delivery device is not fully inserted into the reusable accessory, the time the accelerometer last detected a reversible attachment between the adaptive element(s) and the medication delivery device, and/or that the user has missed a dose of their medication.

In some embodiments, the user interface includes a button, and the attachment sensor(s) can include an accelerometer. In this case, the accelerometer may rest in a low-power mode until the user presses the button to wake the device. Once the user presses the button, the accelerometer switches to a high-sensitivity mode and waits for the signature of the reversible attachment between the adaptive element(s) and the medication delivery device.

In one or more examples, the disclosed devices and methods may include one or more proximity sensors coupled to the housing. The proximity sensor(s) may be located on either end of the housing, radially around the middle, or some combination thereof. In an embodiment, a proximity sensor may be provided on each end of the housing and radially around the middle. The proximity sensor(s) may be used to alert the accelerometer, putting it into a high sensitivity mode. Once in high sensitivity mode, the accelerometer waits for the signature of the reversible attachment between the adaptive element(s) and the medication delivery device.

In one or more examples, the proximity sensor(s) may also detect the type of insulin being inserted into the housing. If the proximity sensor(s) detect that the user is inserting a different type of insulin, the user interface may alert the user of such a change.

In one or more examples, the adaptive element(s) may include interchangeable collars that reversibly couple to the housing, where the interchangeable collars are compatible with different medication delivery devices that have varying geometries.

As referenced herein, “snap fit,” or “snap-fit connection” may refer to a type of fastening mechanism and method to reversibly attach a reusable accessory and a medication delivery device having one end received by the reusable accessory. A snap-fit connection allows the reusable accessory (e.g., a pen cap) and the medication delivery device (e.g., an insulin pen) to be easily assembled or disassembled by applying pressure or snapping them into place. A snap-fit mechanism may include any suitable features such as, e.g., protrusions, undercuts, flexible elements in the components to be joined. For example, a snap-fit may result in a haptic feedback click alerting the user of proper connection.

In one or more examples, the disclosed devices and methods may include interchangeable snap rings that reversibly attach to the housing of the reusable accessory, where the interchangeable rings are compatible with medication delivery devices having varying geometries.

### Example Devices and Methods

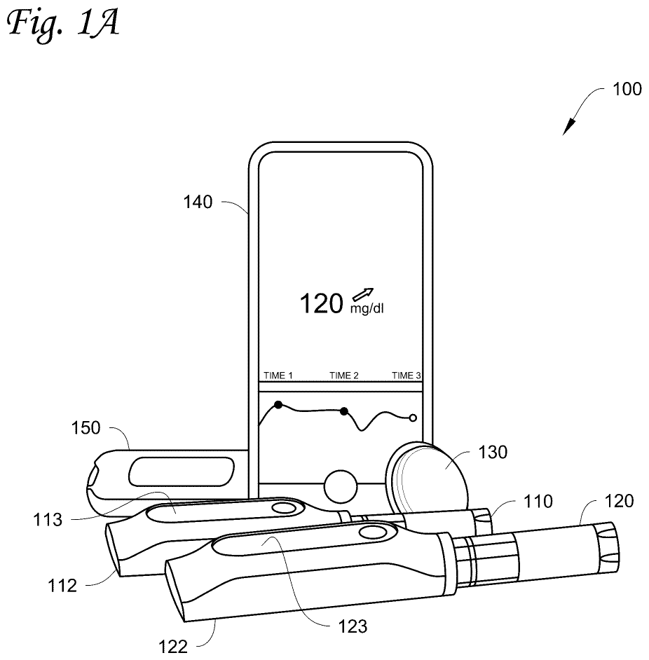

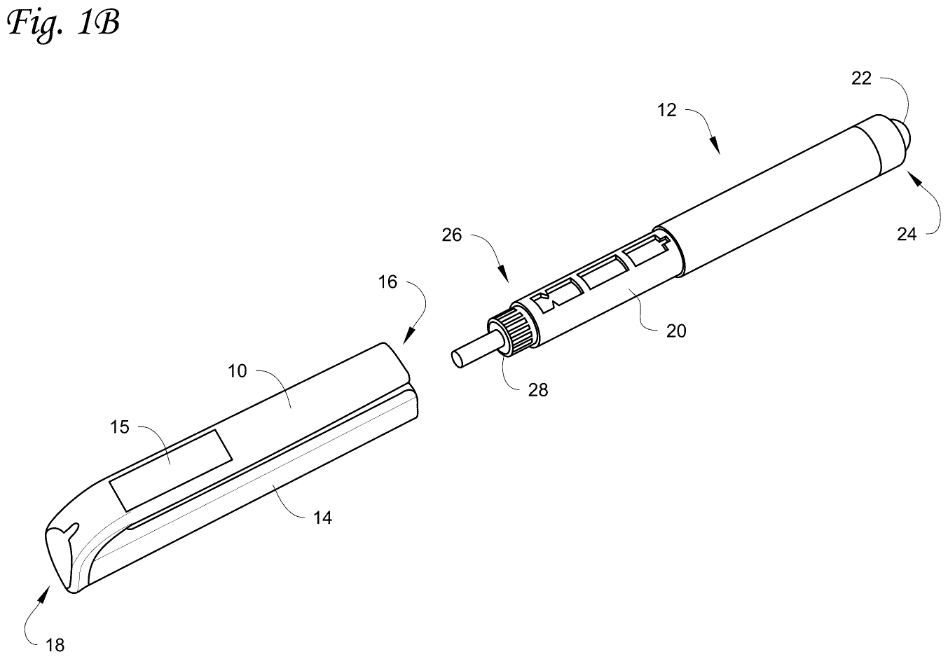

FIG. 1A shows a medication delivery management system 100. The system 100 includes a first medication delivery device 110 having a reusable accessory 112, a second medication delivery device 120 having a reusable accessory 122, a continuous monitor 130, a blood testing meter 150, and a mobile device 140 which has a therapy management mobile application executing thereon.

In the illustrated embodiment, the medication delivery management system 100 may be an insulin therapy management system for delivering insulin (e.g., medication delivered by the system 100 includes insulin). For example, the first medication delivery device 110 is a first insulin pen with the reusable accessory 120 being a first pen cap, the second medication delivery device 110 is a second insulin pen with the reusable accessory 122 being a second pen cap. For example, the continuous monitor 130 is a continuous glucose monitor (“CGM”), the blood testing meter 150 is a blood glucose meter (BGM).

The medication delivery devices 110, 120 (e.g., insulin pens) may include dials (not shown) that may be used to configure the pens to inject a dose of medication (e.g., insulin) that corresponds to the dial turn. The medication delivery devices 110, 120 (e.g., insulin pens) may include interfaces for wireless and/or wired communication with one or more of the pen caps, glucose sensor, mobile devices, and other accessories.

A reuseable accessory (e.g., a pen cap) may include one or more user interface elements for presenting or displaying information and receiving user input. For example, the pen caps 112 and 122 include user interface elements 113 and 123, respectively. The one or more user interface elements 113, 123 may include any suitable hardware, circuitry, logic, firmware and other related components of a pen cap or an associated medication delivery device and configured to support user communications and interactions between the pen cap or the medication delivery device and a user. The one or more user interface elements may include one or more output elements including, for example, a visual output device such as a display, an audio output device such as a speaker, a vibration device, etc. The one or more user interface elements may further include one or more input elements, such as, for example, a button, a knob, a touch screen, or panel, etc.

In some embodiments, pen capping information may be displayed at a user interface element of a pen cap to a user. In some embodiments, pen capping information may be announced by a speaker in the pen cap. For example, in some embodiments, a pen cap may provide a timer clock that counts up (or a timer that counts down) from the last time the pen cap was secured to an injection pen. In some embodiments, a pen cap can wirelessly communicate pen capping information to mobile device 140 (e.g., a smartphone, tablet, etc. running a mobile application). As referenced herein, pen capping information may refer to information about when the pen cap is secured to and/or released from an insulin pen (also referred to herein as “capping” and “uncapping” respectively). Pen capping information may also include information about a current capping period (e.g., the time since the last capping), information about a duration of one or more uncapping, the timing (e.g., time-of-day or time elapsed since) of each uncapping and each capping, etc.

In some embodiments, pen capping information may be used to adjust the user experience. In some embodiments, the pen cap may adjust the presentation of therapy relevant information and/or recommendations provided to the user responsive to the pen capping information. For example, in some embodiments, a pen cap may provide bolus recommendations to correct for elevated glucose levels based on data from the CGM 130, but may limit the presentation of such correction bolus recommendations to time periods when the current pen capping duration is greater than a threshold period of time (e.g., at least 3 hours, at least 4 hours, or at least 5 hours). In some embodiments, the pen caps 112 and 122 may provide notifications, alerts, and/or alarms to the user based on the pen capping information (e.g., based on the amount of time that a pen has been capped and/or uncapped). For example, if the pen caps 112 and 122 are removed from an injection pen within a threshold period of time (e.g., within 30 minutes or 1 hour for a rapid-acting insulin, within 6-12 hours for a long-acting insulin) from a previous capping, the pen cap may provide a visual, audible, and/or tactile notification to indicate that the user may have recently used the pen to administer insulin. In some embodiments, the pen caps 112 and 122 may be in wireless communication with the mobile device 140 and one or more notifications, alerts, and/or alarms based on pen capping information may be announced or displayed on the mobile computing device.

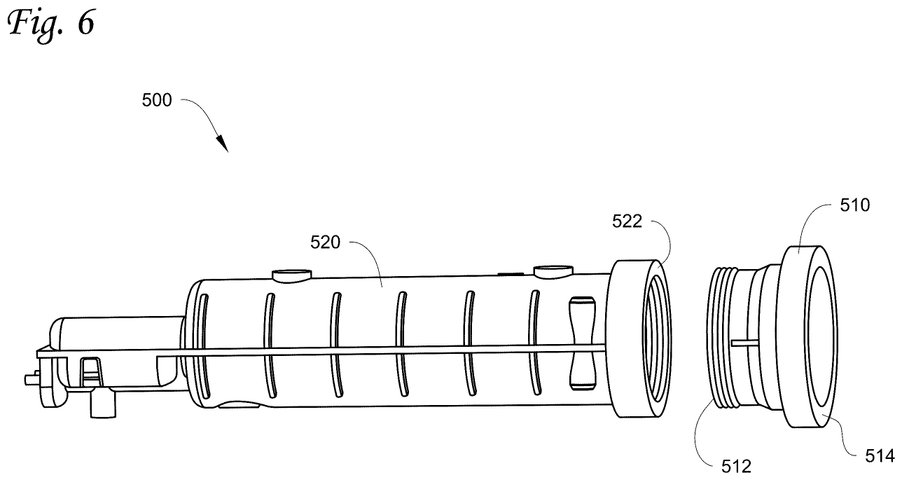

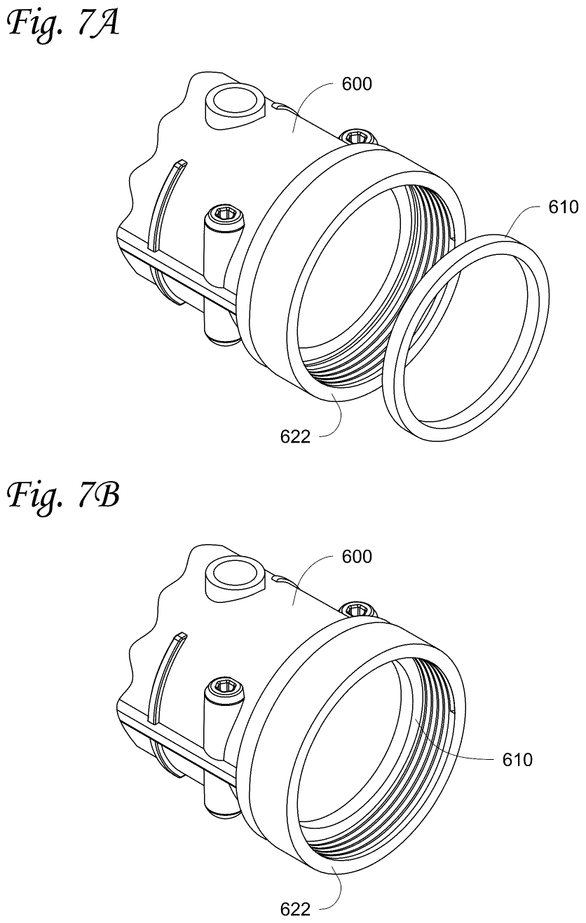

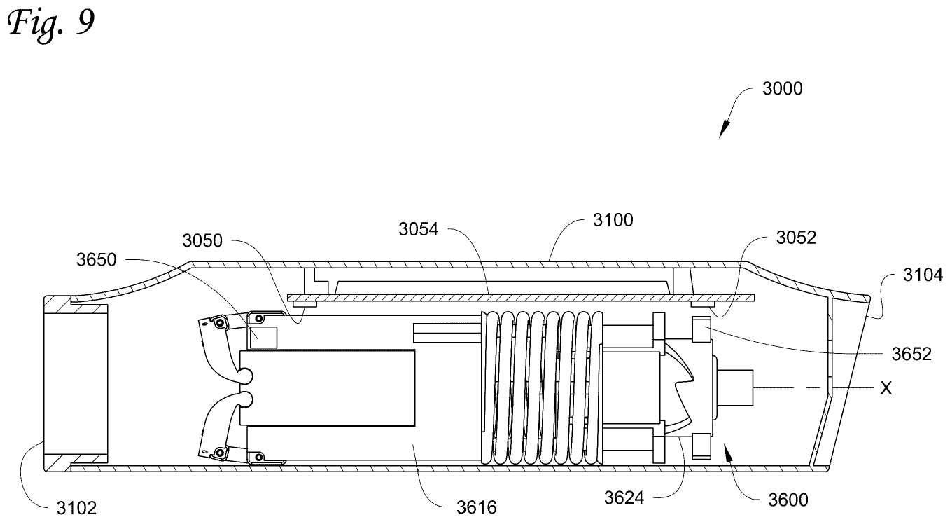

A reusable accessory may include one or more attachment elements configured to adapt the reusable accessory to reversibly couple medication delivery devices having various configurations (e.g., sizes, shapes, geometries, etc.). In some embodiments, the one or more adaptive element(s) may include one or more of a snap ring (e.g., snap ring 610 of FIGS. 7A and 7B), an interchangeable collar (e.g., interchangeable collar 510 of FIG. 6), and/or a clamping mechanism (e.g., clamping mechanism 3600 in FIG. 9). Each of the snap ring and the interchangeable collar can be detachably coupled to the reusable accessory. Each snap ring and interchangeable collar can be configured for the geometry of a specific medication delivery device (e.g., specific geometry of one type/brand/model of insulin pen). In one example, when a reusable accessory is being reused for a medication delivery device that has a different geometry from a previously used medication delivery device, the snap ring and/or interchangeable collar is detached and replaced with a different snap ring and/or interchangeable collar configured for geometry of the medication delivery device with a different geometry.

In one example embodiment, a snap ring or interchangeable collar may form a snap-fit connection with the medication delivery device (e.g., an insulin pen) when the reusable accessory is connected/secured to the medication delivery device.

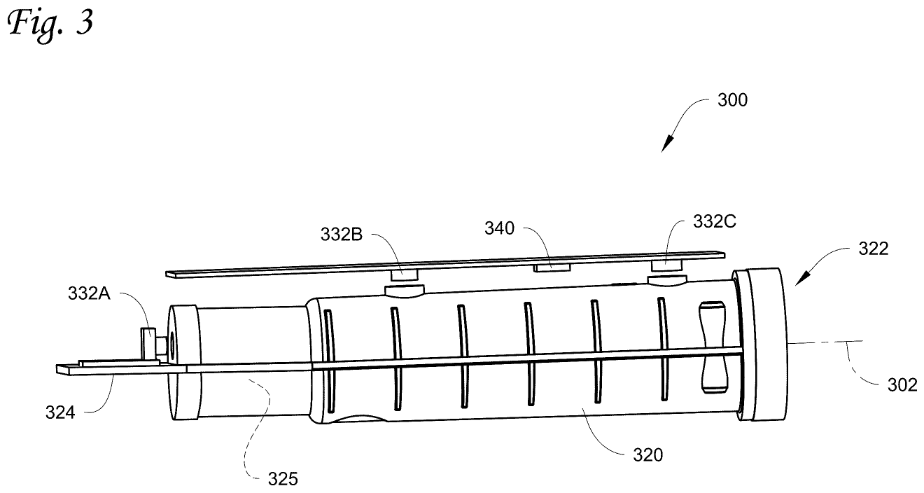

A reusable accessory may include one or more attachment sensors (e.g., one or more of sensors 332A, 332B, 332C,340 in FIG. 3 and sensors 3050, 3052 in FIG. 9) to detect attaching/detaching of the reusable accessory from a medication delivery device, e.g., to detect when the reusable accessory is connected/secured to and/or released/unsecured from the medication delivery device (e.g., to detect capping and/or uncapping of an insulin pen). For example, the reusable accessory can include control circuitry (e.g., printed circuit board 3054, etc.) that receives sensing data from each of the one or more attachment sensors, and is configured to detect attaching/detaching of the reusable accessory from a medication delivery device based on the sensing data from the attachment sensor(s).

In one example embodiment, the reusable accessory may include one or more accelerometers to detect attaching/detaching (e.g., to detect the capping and/or uncapping). A suitable accelerometer may be configured to detect an acceleration, a motion, and/or a vibration of the reusable accessory. In one example, the accelerometer may be configured to detect a snap-fit connection formed between a snap ring or interchangeable collar and a medication delivery device (e.g., an insulin pen). The accelerometer may also detect a motion of the pen cap, e.g., when a PWD picks up the pen cap.

The attachment sensor(s) may include one or more proximity sensors (e.g., proximity sensors 332A, 332B, 332C in FIG. 3) to detect a position (e.g., insertion) of the medication delivery device in the reusable accessory (e.g., an insertion of an insulin pen into the pen cap). In one example embodiment, an array of proximity sensors may be arranged along a longitudinal direction (e.g., a depth direction of the housing of the reuseable direction) to detect an insertion depth of medication delivery device into the reuseable accessory (e.g., of the insulin pen into the pen cap). When the proximity sensors detect the insertion of a medication delivery device to a certain depth of the reuseable accessory, a different type of sensor (e.g., the accelerometer) can enter into a high-sensitivity mode to detect for a reversible attachment (e.g., a snap-fit connection) formed between the medication delivery device and the reusable accessory. A suitable proximity sensor can include any sensing mechanisms to detect the location of the medication delivery device (e.g., a pen tip of an inulin pen) inside a reusable accessory (e.g., inside the pen cap). An example proximity sensor can include an optical proximity sensor (e.g., an infrared proximity sensor), a pressure sensor, etc.

A reusable accessory may further include a communications interface (not shown) which may include any suitable hardware, circuitry, logic, firmware and other related components of the reusable accessory that are coupled to the control circuitry and configured to support communications between the pen cap and a medication delivery device, between the pen cap and one or more external electronic devices, mobile computing devices, and a cloud networking such as a network. In some cases, the communication interface may use one or more wireless communication techniques such as, for example, near-field communication (NFC), Bluetooth low energy (BLE), Wi-Fi, etc. In some embodiments, the communications interface may also support wired communications.

FIG. 1B shows a perspective view of a reusable accessory 10 (e.g., a pen cap) for a medication delivery device 12 (e.g., a pen-shaped medication delivery device, an insulin pen). The reusable accessory 10 includes an elongated hollow housing 14 with a first open end 16 and a second closed end 18 opposite the first open end 16. The reusable accessory 10 further includes a user interface element 15 on the pen cap 10.

In this embodiment, the medication delivery device 12 is a disposable insulin pen that includes a reservoir 20 in which liquid insulin is held, a plunger 22 at a back end 24 of the insulin pen, and a hypodermic needle (not shown) from which the insulin is closed at the tip 28 of a front part 26 of the insulin pen. It should be appreciated that in a different embodiment, the medication delivery device 12 may be configured to deliver a liquid medication different from insulin, in which the reservoir 20 contains a liquid medication other than insulin. The first open end 16 of the cap 10 can be placed over the front part 26 of the medication delivery device 12 so that the reusable accessor 10 fits onto and covers the front part 26 of the medication delivery device 12. For example, as shown in FIG. 1B, the medication delivery device 12 can be an insulin pen, and the reusable accessory can be a pen cap.

It is to be understood that while the embodiments of FIGS. 1A-B illustrate insulin pens with the respective pen caps, the medication delivery device 12 is not limited to insulin pens and can be a different type of medication delivery device (e.g., a different medication delivery device having a pen shape), and the reusable accessory (e.g., a cap) can be provided, as a kit or independently, for receiving/covering at least an end portion of the medication delivery device.

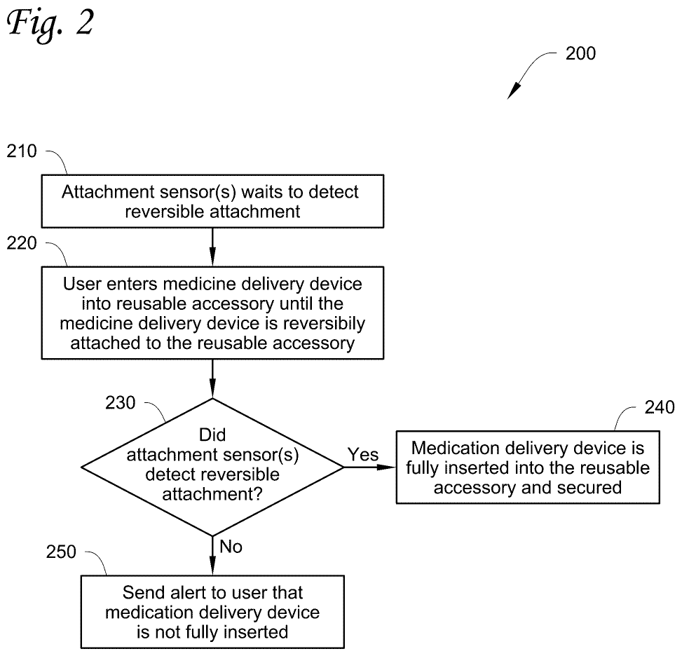

FIG. 2 illustrates a block flow chart of a method 200 for detecting reversible attachment of a medication delivery device to a reusable accessory. For example, the method 200 may be used to detect capping of the medication delivery device (e.g., insulin pen) with the reusable accessory (e.g., a pen cap). The reusable accessory (e.g., pen cap) includes an accelerometer to detect reversible attachment (e.g., a snap-fit connection) between an adaptive element(s) of the reusable accessory and the medication delivery device (e.g., an insulin pen).

In this example, at block 210, the accelerometer is in a high-sensitivity mode and waits to detect a reversable attachment (e.g., a snap-fit). At block 220, the user enters (e.g., inserts) the medication delivery device into the housing until the medication delivery device makes a reversable attachment with the adaptive element(s) of the reusable accessory (e.g., with a snap ring). At block 230, a controller determines whether the accelerometer detects a reversable attachment between the adaptive element(s) and the medication delivery device. When the accelerometer detects the signature of the reversable attachment (e.g., the signature for a snap-fit), the method 200 proceeds to block 240 at which a user interface may indicate that the medication delivery device is fully inserted into the reusable accessory. In some embodiments, the detection of the proper attachment (e.g., of the reversable attachment occurring) may start a clock that counts down until a next dose. When the accelerometer does not detect the signature of the reversable attachment, the method 200 proceeds to block 250 at which the user interface may send an alert to the user that the medication delivery device is not fully inserted.

### Example Device Including Proximity Sensors

FIG. 3 shows a side view of a reusable accessory 300, according to an embodiment. As shown in the illustrated embodiment, the reusable accessory 300 can be a pen cap. The reusable accessory 300 includes a housing 320 extending along a housing axis 302 between a distal end 322 and a proximal end 324. The reusable accessory 300 includes multiple proximity sensors 332A, 332B, 332C disposed along the housing 320 of the reusable accessory 300. In the depicted embodiment, a first proximity sensor 332A (PS1) is located at the closed, proximal end 324 of the housing 320. A second proximity sensor 332B (PS2) is located radially around the middle of the housing 320. A third proximity sensor 332C (PS3) is located at the open, distal end 322 of the housing 320.

While three proximity sensors are illustrated in the embodiment of FIG. 3, it is to be understood that the reusable accessory 300 in other embodiments may include any suitable numbers of proximity sensors 332A, 332B, 332C disposed at various positions of the reusable accessory 300. The placement of the proximity sensors along the housing 320 can be determined by desired applications. In the embodiment depicted in FIG. 3, the proximity sensors 332A, 332B, 332C are positioned along a length direction of the housing 320 to detect an insertion depth of a medication delivery device (e.g., insulin pen) into the housing 320 of the reusable accessory 300.

The reusable accessory 300 further includes an accelerometer 340 (A1) disposed on the housing 320 between the proximity sensor 332B (PS2) and the proximity sensor 332C (PS3). The accelerometer 340 is for detecting attachment/detachment (e.g., the capping and/or uncapping). A suitable accelerometer may be configured to detect an acceleration, a motion, and/or a vibration of the reusable accessory 300. For example, the accelerometer 340 may be configured to detect a reversable attachment (e.g., a snap-fit attachment) formed between an adaptive element (e.g., a snap ring or interchangeable collar) and a medication delivery device (e.g., an insulin pen).

The accelerometer may also detect a motion of the reusable accessory 300, e.g., when a PWD picks up the reusable accessory 300. The one or more proximity sensors can detect an insertion depth of a medication delivery device (e.g., an insulin pen) into the reusable accessory 300. In one example embodiment, when the proximity sensor(s) detect the insertion of an injection pen into a certain depth of the reusable accessory 300, the accelerometer can enter into a high-sensitivity mode to detect a snap-fit attachment between the reusable accessory 300 and the medication delivery device.

In the embodiment depicted in FIG. 3, the accelerometer 340 is disposed on the outside of the housing 320. It is to be understood that the accelerometer 340 may be received or supported by the housing 320 at other suitable locations as long as the accelerometer can detect a secured attachment (e.g., a snap-fit attachment between the reusable accessory 300 and the medication delivery device). The accelerometer 340 may be located to avoid any direct contact with the medication delivery device which may cause damage to the accelerometer with the direct contact, in particular, when the medication delivery device has a needle mounted on a tip of the medication delivery device (e.g., a needle of an insulin pen).

It is to be understood that the housing 320 may be configured to receive medication delivery devices (e.g., insulin pens) and needles with various sizes. As shown in FIG. 3, the housing 320 may have an internal space 325 adjacent to the closed end 324 to receive the needle mounted on the medication delivery device. When a sensor (e.g., an actuator) is located inside the housing 320 to interact with the inserting pen to detect the capping, the interaction may cause damage to the sensor (e.g., an actuator) inside the housing 320. The accelerometer 340 can detect capping without a direct contact with the inserting pen and can be located at any desired locations to avoid the direct contact.

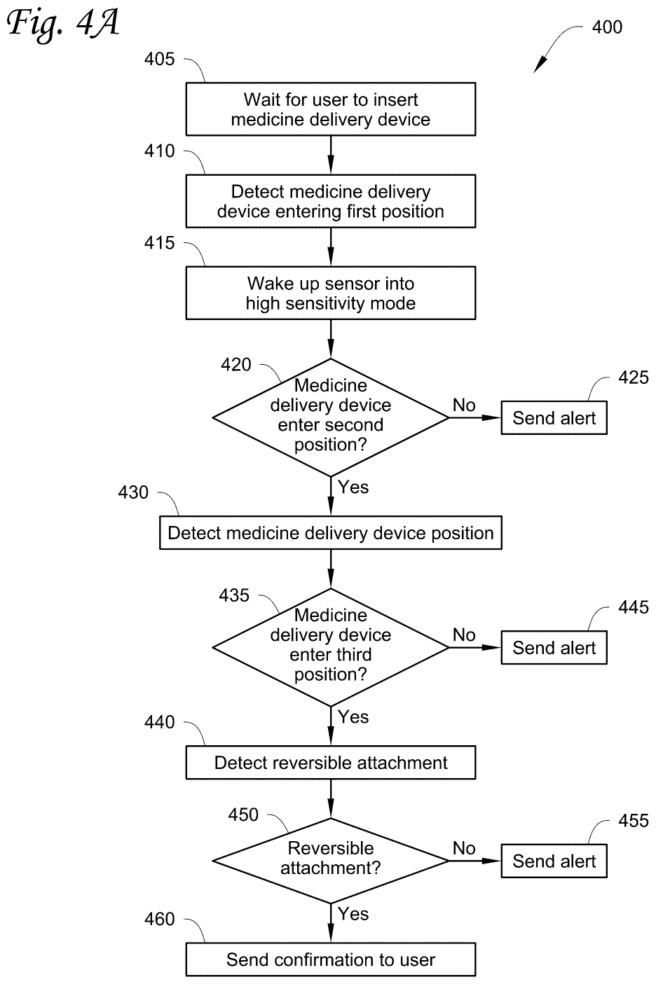

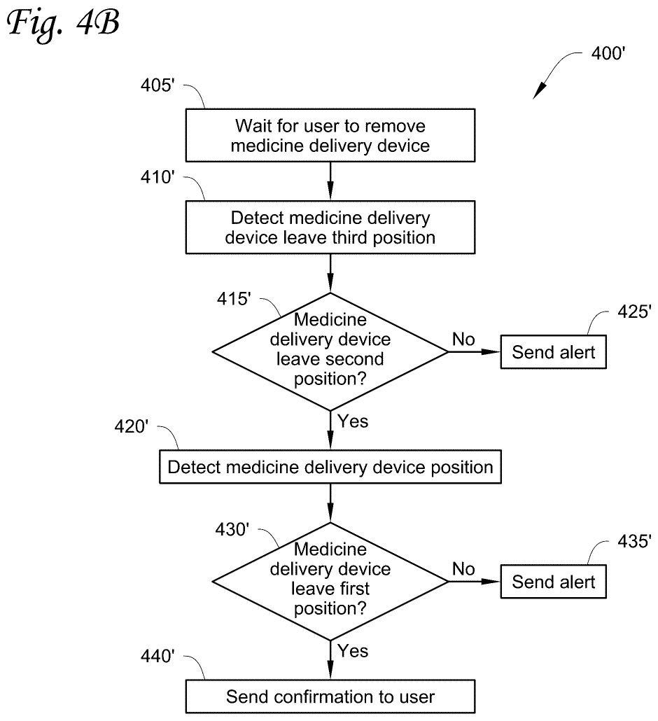

FIG. 4A illustrates a flow chart of a method 400 for detecting reversable attachment of a medication delivery device in a reusable accessory. One or more proximity sensors (e.g., the proximity sensors 332A, 332B, 332C of FIG. 3) can be used to put one or more accelerometers (e.g., the accelerometer 340 of FIG. 3) into a high-sensitivity mode and to detect proper attachment between a snap ring connected to a pen cap and a medication delivery device (e.g., an insulin pen).

It is to be understood that the method 400 disclosed herein may be conducted by one or more processors or controllers, and/or one or more components of a pen cap or a medication delivery device connected to the pen cap unless otherwise specified. The conducting processor/controller may communicate with other components of a medication delivery system (e.g., the medication delivery system 100 of FIG. 1A). It is also to be understood that the method 400 may include one or more operations, actions, or functions as illustrated by one or more of blocks 405, 410, 415, 420, 425, 430, 435, 440, 445, 450, 455, and 460. These various operations, functions, or actions may, for example, correspond to software, program code, or program instructions executable by a processor/controller, hardware, or a combination thereof that causes the functions to be performed. Although illustrated as discrete blocks, obvious modifications may be made, e.g., two or more of the blocks may be re-ordered; further blocks may be added; and various blocks may be divided into additional blocks, combined into fewer blocks, or eliminated, depending on the desired implementation. The method 400 may begin at block 405.

At block 405 (Wait for user to insert medication delivery device), a controller can control the reusable accessory to be in a standby mode, which waits for a user to begin to insert a medication delivery device (e.g., an insulin pen) into the housing of the reusable accessory. The method 400 may proceed from block 405 to block 410.

At block 410 (Detect medication delivery device entering first position), the controller determines, based on the sensing data from one or more proximity sensors, that the medication delivery device enters a first position within the housing of the reusable accessory. For example, the proximity sensor 332C (PS3) can detects that a tip of the medication delivery device (e.g., a pen tip of an insulin pen) enters the housing of the reusable accessory, e.g., being adjacent to the proximity sensor 332C (PS3) at the first position within the housing of the reusable accessory. The method 400 may proceed from block 410 to block 415.

At block 415 (Wake-up accelerometer into high sensitivity mode), the controller can send a control signal to wake-up the accelerometer 340 (A1) and set the accelerometer 340 (A1) into a high sensitivity mode. For example, the accelerometer 340 (A1) can be in a standby mode to save energy until the controller determines that an inserting pen inserts into a certain depth of the pen cap. That is, the accelerometer 340 (A1) may not be activated until the moment when the inserting pen inserts into the pen cap. The method 400 may proceed from block 415 to block 420.

At block 420 (Medication delivery device enter second position?), the controller determines, based on the sensing data from one or more proximity sensors, whether the medication delivery device enters a second position of the housing of the pen cap. For example, the proximity sensor 332B (PS2) can detect that the medication delivery device reaches its threshold within a specified amount of time and send the sensing data to the controller to determine whether the medication delivery device enters the second position. When the controller determines that the medication delivery device does not reach the proximity sensor 332B (PS2) within a specified amount of time, the method 400 may proceed from block 420 to block 425. When the controller determines that the medication delivery device does reach the proximity sensor 332B (PS2) within a specified amount of time, the method 400 may proceed from block 420 to block 430.

At block 425 (Send alert), the controller can send a control signal to a user interface to send an alert to the user that the medication delivery device is not fully inserted in the reusable accessory.

At block 430 (Detect medication delivery device position), the controller can receive sensing data from the proximity sensor 332A (PS1) to determine a position of the medication delivery device inside the housing of the reusable accessory. The method 400 may proceed from block 430 to block 435.

At block 435 (Medication delivery device enter third position?), the controller determines, based on the sensing data from one or more proximity sensors, whether the medication delivery device enters a third position of the housing of the reusable accessory. For example, the proximity sensor 332A (PS1) can detect that the medication delivery device reaches its threshold within a specified amount of time and send the sensing data to the controller to determine whether the medication delivery device enters the third position. When the controller determines that the medication delivery device does not reach the proximity sensor 332A (PS1) within a specified amount of time, processing may proceed from block 435 to block 445. When the controller determines that the medication delivery device does reach the proximity sensor 332A (PS1) within a specified amount of time, the method 400 may proceed from block 435 to block 440.

At block 440 (Detect connection), the accelerometer 340 (A1) can detect the signature of a reversible attachment (e.g., a snap-fit attachment) between the pen cap and the medication delivery device, and send the sensing data to the controller. In some embodiments, the signature of a snap-fit connection can be a sudden spike in acceleration from the pen “clicking in” and then a sudden deceleration when the “clicking” is complete and the reusable accessory and medication delivery device combination are at rest. The method 400 may proceed from block 440 to block 445.

At block 445 (Send alert), the controller can send a control signal to a user interface to send an alert to the user that the medication delivery device is not fully inserted (e.g., is not securely attached to the reusable accessory). At block 450 (Reversible attachment?), the controller can determine, based on the sensing data from the accelerometer 340 (A1), whether the reversible attachment is formed between the reusable accessory and the medication delivery device. When the controller determines that a reversible attachment is formed between the reusable accessory and the medication delivery device, the method 400 may proceed from block 450 to 460. When the controller determines that a reversible attachment is not formed between the reusable accessory and the medication delivery device, the method 400 may proceed from block 450 to 455.

At block 455 (Send alert), the controller can send a control signal to a user interface to send an alert to the user that the medication delivery device is not properly connected to the housing of the reusable accessory.

At block 460 (Send confirmation to user), the controller can send a control signal to the user interface to send a notification/confirmation to the user that the medication delivery device is fully inserted and secured in the housing of the reusable accessory.

FIG. 4B illustrates a block flow chart of a method 400′ for detecting a removal of a medication delivery device from a reusable accessory. The method 400′ can detect the removal of the medication delivery device from the housing of the reuseable accessory. For example, the method 400′ in an embodiment may be used to detect removal of a medication delivery device that is an insulin pen from a reusable accessory that is a pen cap.

It is to be understood that the method 400′ disclosed herein may be conducted by one or more processors or controllers, and/or one or more components of a reusable accessory or a medication delivery device connected to the reusable accessory unless otherwise specified. The conducting processor/controller may communicate with other components of a medication delivery system (e.g., the medication delivery system 100 of FIG. 1A). It is also to be understood that the method 400 may include one or more operations, actions, or functions as illustrated by one or more of blocks 405′, 410′, 415′, 420′, 425′, 430′, 435′, and 440′. These various operations, functions, or actions may, for example, correspond to software, program code, or program instructions executable by a processor/controller, hardware, or a combination thereof that causes the functions to be performed. Although illustrated as discrete blocks, obvious modifications may be made, e.g., two or more of the blocks may be re-ordered; further blocks may be added; and various blocks may be divided into additional blocks, combined into fewer blocks, or eliminated, depending on the desired implementation. The method 400′ may begin at block 405′.

At block 405′ (Wait for user to remove medication delivery device), a controller can control the reusable accessory being in a standby mode, waiting for a user to begin to remove the medication delivery device (e.g., insulin pen) from the housing of the reusable accessory (e.g., pen cap). The method 400′ may proceed from block 405′ to block 410′.

At block 410′ (Detect medication delivery device leave third position), the controller determines, based on the sensing data from one or more proximity sensors, that the medication delivery device leaves the third position of the housing of the reusable accessory. For example, the controller can determine, based on the sensing data from the proximity sensor 332A (PS1) that the medication delivery device leaves the threshold of the proximity sensor 332A (PS1). The method 400′ may proceed from block 410′ to block 415′.

At block 415′ (Medication delivery device leave second position?), the controller determines, based on the sensing data from one or more proximity sensors, whether the medication delivery device leaves the second position of the housing of the reusable accessory. For example, the controller may determine, based on the sensing data from the proximity sensor 332B (PS2), whether the medication delivery device reaches the threshold of the proximity sensor 332B (PS2) within a specified amount of time. When the controller determines that the medication delivery device does reach proximity sensor 332B (PS2) within a specified amount of time, processing may proceed from block 415′ to block 420′. When the controller determines that the medication delivery device does not reach proximity sensor 332B (PS2) within the specified amount of time, method 400′ may proceed from block 415′ to block 425′.

At block 420′ (Detect medication delivery device position), the controller can receive the sensing data from the proximity sensor 332C (PS3) to determine a position of the medication delivery device inside the housing of the reusable accessory. The method 400′ may proceed from block 420′ to block 430′.

At block 430′ (Medication delivery device leave first position?), the controller determines whether the medication delivery device leaves the first position within the housing of the reusable accessory. For example, when controller determines, based on the sensing data from the proximity sensor 332C (PS3), the medication delivery device reaches the proximity sensor 332C (PS3) within a specified amount of time, processing may proceed from block 430′ to block 440′. When controller determines, based on the sensing data from the proximity sensor 332C (PS3), the medication delivery device does not reach the proximity sensor 332C (PS3) within a specified amount of time, the method 400′ may proceed from block 430′ to block 435′.

At block 435′ (Send alert), the controller can send a control signal to the user interface to send an alert to the user that the medication delivery device is not properly inserted.

At block 440′ (Send confirmation), the controller can send a control signal to the user interface to send a confirmation/notification to the user that the medication delivery device has been fully removed from the housing of the reusable accessory.

It should be appreciated that the method 400 in FIG. 4A and/or the method 400′ in FIG. 4B may be applied to the sensing of the removable attachment of different adaptive elements to a medication delivery device. For example, the method 400 in FIG. 4A and/or the method 400′ in FIG. 4B in other embodiments may be applied to the sensing of the adaptive element removably attaching to a medication delivery device in FIGS. 9-12 as discussed below. For example, in some embodiments, proximity sensor(s) (e.g., the proximity sensors 332A, 332B, 332C) as discussed above may modified to change the attachment sensor(s) 3050, 3052 in the reusable accessory 3000 in FIGS. 9-12 from a standby mode to a high sensitivity mode as discussed in the method 400. For example, in some embodiments, the sending of alert(s)/confirmation(s) as discussed in the method 400 and/or method 400′ may be incorporated into and/or employed by the reusable accessory 3000.

### Example Reuseable Accessory Adaptive Element Including Interchangeable Collar(s) and/or Snap Ring(s)

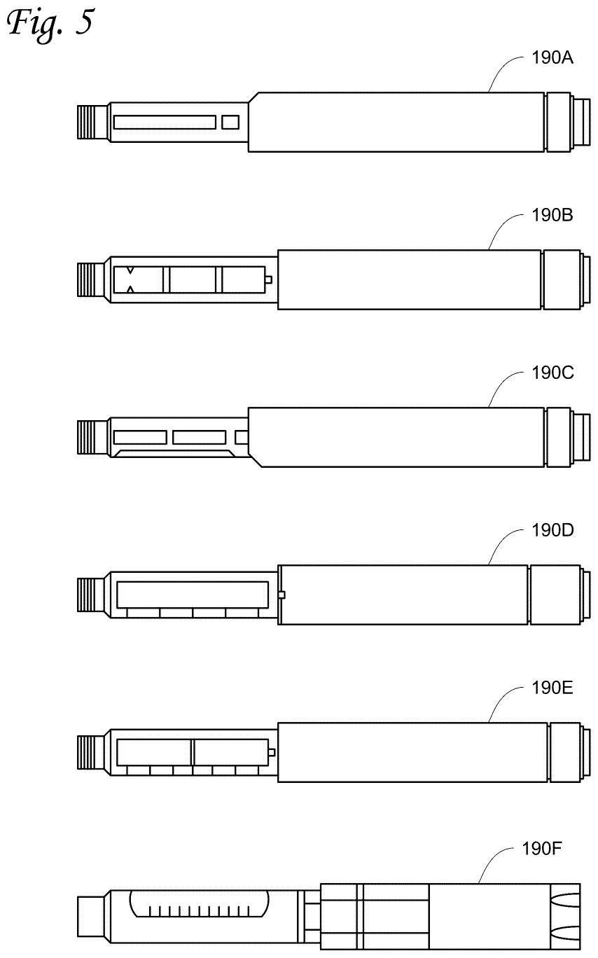

FIG. 6 shows a reusable accessory 500 with an adaptive element separated from the reusable accessory 500, according to an embodiment. The reusable accessory 500 includes a housing 520 for receiving a medication delivery device. In the illustrated embodiment of FIG. 5, the adaptive element is an interchangeable collar 510. It should be appreciated that features discussed for the interchangeable collar 510 may generally apply to the adaptive element in an embodiment.

The interchangeable collar 510 has a first end 512 and a second end 514 opposite to the first end 512. The first end 512 of the interchangeable collar 510 is coupled to, a first end 522 of the housing 520 of the reusable accessory 500 to form a connection. For example, as shown in FIG. 6, the first end 512 of the interchangeable collar 510 couples to the first end 522 of the housing 520 by screwing onto the first end 522. It should be appreciated that the reversible coupling of the interchangeable collar 510 to the housing 520 may be via a different manner in other embodiments. For example, interchangeable collars in an embodiment may be configured to couple to the housing 520 via a lock and key mechanism. For example, interchangeable collars in an embedment may be configured to couple to the housing via a snap-fit.

The interchangeable collar 510 is configured to reversibly attach to, via the second end 514, a medication delivery device. In an embodiment, the interchangeable collar 510 may be configured to reversibly attach to a medication delivery device (e.g., one of the medication delivery devices 110, 120 in FIG. 1A, one of the medication delivery devices 190A-190F in FIG. 4). In an embodiment, the reusable accessory 500 may use a different interchangeable collar (not shown) (e.g., a second one of the interchangeable collars 710 in FIG. 7) for receiving a medication delivery device with a different geometry (e.g., a second one of the medication delivery devices 110, 120 in FIG. 1A, the medication delivery device 12 in FIG. 1B, a second one of the medication delivery devices 190A-190F in FIG. 4). In an embodiment, the reusable accessory 500 without the interchangeable collar 510 may be configured to receive the medication delivery device with a different geometry. In the illustrated embodiment, the reusable accessory 500 is a pen cap, and the medication delivery device(s) are insulin pens (e.g., the medication delivery devices 110, 120 in FIG. 1A, the medication delivery devices 190A-190F in FIG. 4).

FIG. 7A shows a reusable accessory 600 with an adaptive element separated from the reusable accessory 600, according to an embodiment. FIG. 7B shows the adaptive element reversibly coupled to the reusable accessory 600. The reusable accessory 600 includes a housing 620 for receiving a medication delivery device. In the illustrated embodiment of FIGS. 6A and 6B, the adaptive element is an interchangeable ring 610. It should be appreciated that features discussed for the interchangeable ring 610 may generally apply to the adaptive element in an embodiment.

As shown in FIG. 7B, the interchangeable ring 610 is coupled to the housing 620 of the reusable accessory 600 to form a connection. In particular, the interchangeable ring 610 is coupled to the first end 622 of the housing 620 of the reusable accessory 600. For example, the interchangeable ring 610 is inserted into the housing 620 of a reusable accessory 600 prior to the insertion of a medication delivery device, according to an embodiment. The interchangeable ring 610 couples to the housing 620 by fitting into the first end 622 of the housing 620 (e.g., friction fit into the housing 620, etc.).

The interchangeable ring 610 is configured to reversibly attach to the medication delivery device (e.g., one of the medication delivery devices 110, 120 in FIG. 1A, one of the medication delivery devices 190A-190F in FIG. 4). In the illustrated embodiment, the interchangeable ring 610 is configured to reversibly attach to the medication delivery device by fitting to the medication delivery device. For example, the interchangeable ring 610 can be a snap ring shaped so as to snap on to (e.g., around) the medication delivery device received in the reusable accessory 600. In an embodiment, the reusable accessory 600 may use a different interchangeable ring (not shown) for receiving a medication delivery device with a different geometry (e.g., a second one of the medication delivery devices 110, 120 in FIG. 1A, the medication delivery device 12 in FIG. 1B, a second one of the medication delivery devices 190A-190F in FIG. 4). In the illustrated embodiment, the reusable accessory 500 is a pen cap, and the medication delivery device(s) are insulin pens (e.g., the medication delivery devices 110, 120 in FIG. 1A, the medication delivery devices 190A-190F in FIG. 4).

In an embodiment, an adaptive element for a reusable accessory (e.g., reuseable accessory 500, reuseable accessory 500) may include both an interchangeable collar (e.g., interchangeable collar 510) and an interchangeable ring (e.g., the interchangeable ring 610). In one example, the interchangeable ring is coupled (e.g., fit into) the housing of the reuseable accessory (e.g., a distal end of the housing), and the interchangeable collar is coupled onto the housing with the interchangeable ring disposed between the housing and the interchangeable collar. The interchangeable collar and the interchangeable ring can each be configured to reversible couple to a medication delivery device received in the housing of the reusable accessory.

### Example Reusable Accessory Adaption Kit

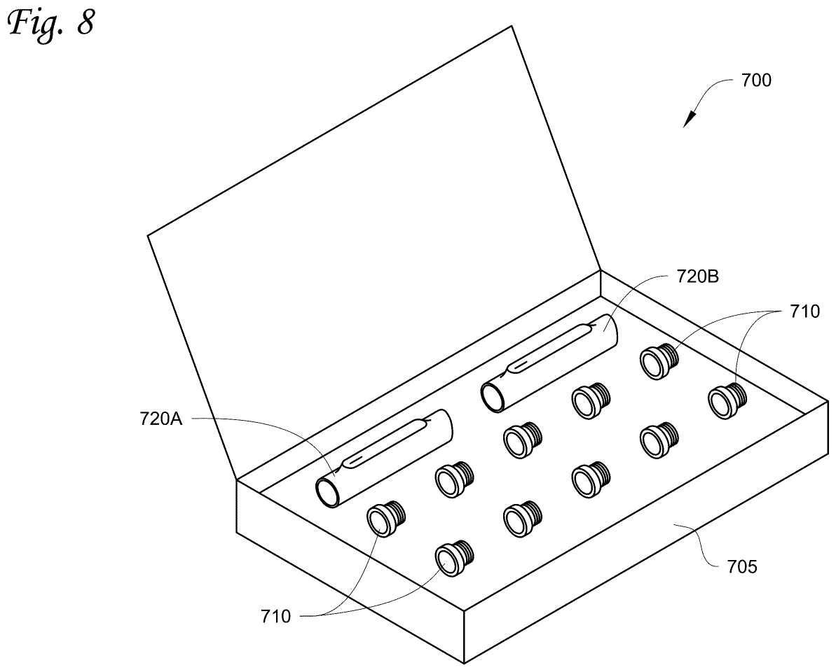

FIG. 8 shows a kit 700 including a plurality of reusable accessories 720A, 720B and a plurality of adaptive elements 710 disposed in a container 705 (e.g., a box, bag, etc.). Each of the adaptive elements 710 is compatible with the reusable accessories 720A, 720B and a medication delivery device (e.g., medication delivery devices 110, 120 in FIG. 1A, medication delivery devices 190A-190B in FIG. 5). For example, the reusable accessories 720A, 720B can be pen caps for use with an insulin pen (e.g., the insulin pens 110 and 120 of FIG. 1A, and the insulin pens 190A-190F with varying geometries in FIG. 5), as shown in the illustrated embodiment. The adaptive elements 710 can have different dimensions for the different geometries of different medication delivery devices. For example, the adaptive elements 710 in the kit 710 can include adaptive elements for at least two medication delivery devices with different geometries.

The adaptive elements 720A, 720B can include a plurality of interchangeable collars, in the illustrated embodiment shown as shown in FIG. 8. It should be appreciated that the adaptive elements 720A, 720B in another embodiment may be/include a plurality of interchangeable rings or a combination of one or more interchangeable rings and one or more interchangeable collars.

While two reusable accessories 720A, 720B and ten adaptive elements 710 are illustrated in the embodiment of FIG. 8, it is to be understood that the kit 700 in other embodiments may include any suitable number of reusable accessories 720A, 720B and adaptive elements to be connected to the reusable accessories 720A, 720B. For example, the kit 700 in an embodiment may include one or more reusable accessories 720A, 720B and two or more adaptive elements 710 for adapting the one or more reusable accessories 720A, 720B for receiving any of a plurality of medication delivery devices. For example, the kit 700 in an embodiment may include two or more adaptive elements 710 for each of the one or more reusable accessories 720A, 720B in the kit 700 to allow each reuseable accessory 710A, 710B to be adaptable to a plurality of geometries of medication delivery devices (e.g., two of the medication delivery devices 190A-190B in FIG. 5).

### Example Reuseable Accessory Adaptive Element With Clamping Mechanism

FIG. 9 illustrates a side sectional view of an embodiment of a reusable accessory 3000. The reusable accessory 3000 includes a housing 3100 configured to receive a medication delivery device 3001 (shown in FIGS. 11A-1-11F-1). The medication delivery device 3001 may be one of the medication delivery devices 110, 120 in FIG. 1A and the medication delivery devices 190A-190B as shown in FIG. 5 with different geometries.

The reusable accessory 3000 includes a distal end 3102 and a proximal end 3104 opposite to the distal end 3102. The housing 3100 extends along a housing axis X between the distal end 3102 and the proximal end 3104. For example, the distal end 3102 and the proximal end 3104 are also the distal end and the proximal end of the housing 3100. The medication delivery device is inserted into the housing 3100 through the distal end 3104.

The reusable accessory 3000 includes an adaptive element coupled to the housing 3100. The adaptive element is configured to reversibly coupled to the medication delivery device (e.g., an insulin pen). The adaptive element can include a clamping mechanism 3600 configured to clamp onto the medication delivery device received in the housing 3100. In the illustrated embodiment, the medication delivery device can be an insulin pen, and the reuseable accessory 3000 can be a pen cap, as shown in the illustrated embodiment of FIGS. 9-11F-1.

The reusable accessory 3000 can also include attachment sensors 3050, 3052 configured to detect the reversible attachment of the adaptive element (e.g., clamping mechanism 3600) to a medication delivery device. The sensors 3050, 3052 are disposed within the housing 3100. As shown in FIG. 9, the attachment sensors 3050, 3052 may each be mounted to a printed circuit board 3054. In other embodiments, one or more of the attachment sensors 3050, 3052 may be mounted to the housing 3100 separate from the printed circuit board 3054. The attachment sensors 3050, 3052 are discussed in more detail below.

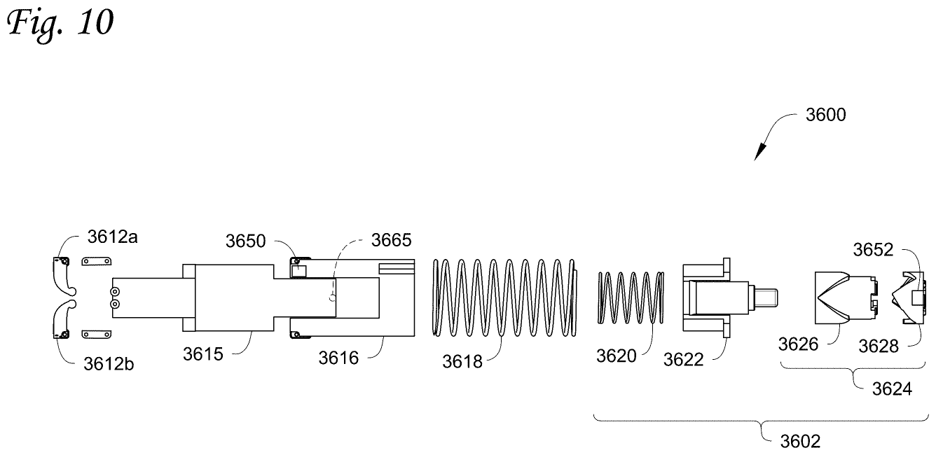

FIG. 10 is an exploded view of the clamping mechanism 3600 of the reuseable accessory 3000, according to an embodiment. The clamping mechanism 3600 includes a clicker mechanism 3902. The clicker mechanism 3602 includes a spring 3618, a clicker retainer 3622, and a clicker portion 3624 made up of a first clicker body 3626 and a second clicker body 3628. The first and second clicker bodies 3624, 3626 are configured to form a guide path 3905 that wraps around the clicker bodies 3624, 3626.

The clamping mechanism 3600 includes clamps 3612a,b, a spring well 3616, a datum 3615, and a clamp spring 3618 attached at one end to the spring well 3616. In some embodiments, the clamps 3612a,b are attached to the spring well 3616 via a hinge connection. Accordingly, the clamps 3612a,b are configured to swing radially inward toward a center longitudinal axis (e.g., the housing axis X) of the reusable accessory 3000 and engage (e.g., contact) the medication delivery device as the medication delivery device is inserted into the reusable accessory 3000. In response to the clamps 3612a,b swinging radially inward, the spring well 3616 is pushed along the longitudinal axis and compresses the clamp spring 3618.

In some embodiments, at least a portion of the datum 3615 is encompassed within the clamp spring 3618. The datum 3615 is configured to move along the longitudinal axis of the reusable accessory 3000 within the clamp spring 3618 in engagement with the clicker mechanism 3602. Longitudinal movement of the datum 3615 occurs upon insertion of the medication delivery device through the spring well 3616 and into and through the datum 3615. In some embodiments, the datum 3615 is shaped to allow at least an end portion of the medication delivery device (e.g., a portion of the needle and/or needle cover) to pass there through. The datum 3615 includes two longitudinally extending arms that form an opening that allows components of the clicker mechanism 3602 to pass therethrough. The datum 3615 includes a protrusion 3670 that protrudes radially inward of the datum 3615 and positioned along the longitudinal length of the datum 3615 that is configured to ride along a guide path 3905 of the clicker mechanism 3602. In some embodiments, each of the longitudinally extending arms of the datum 3615 includes a protrusion 3665 that protrudes radially inward. Each of the protrusions 3665 is configured to ride along the guide path 3905.

The datum 3615 is configured to move forward and backward along the longitudinal axis of the reuseable accessory 3600 over the bias member 3620, the clicker retainer 3622 and the first clicker body 3624. The linear travel of the datum 3615 along the longitudinal axis of the reuseable accessory 3600 causes the inwardly protruding protrusion(s) 3665 to travel along the guide path 3905 formed by the first and second clicker bodies 3624, 3626 and push the first and second clicker bodies 3624, 3626 to rotate the first and second clicker bodies 3624, 3626 and to compress the bias member 3620. The compressed bias member 3620 then pushes the datum 3615 backward (by a relatively slight length) such that the inwardly protruding protrusion(s) 3665 is stably positioned in the guide path 1905.

In an embodiment, a reuseable element may include one or more adaptive elements with features as described for the adaptable elements in U.S. application Ser. No. 18/398,051, which is incorporated herein by reference. For example, in some embodiments, the clamping mechanism 3600 and/or the clicker mechanism 3602 in the reusable accessory may various features as described in U.S. application Ser. No. 18/398,051 for a clamping mechanism and a clicker mechanism, respectively.

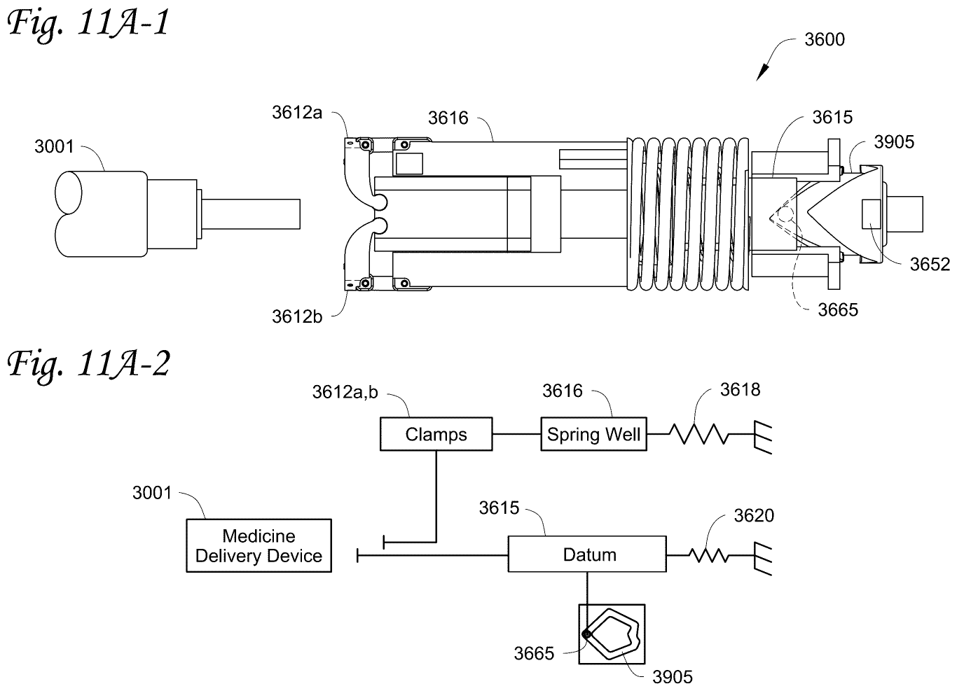

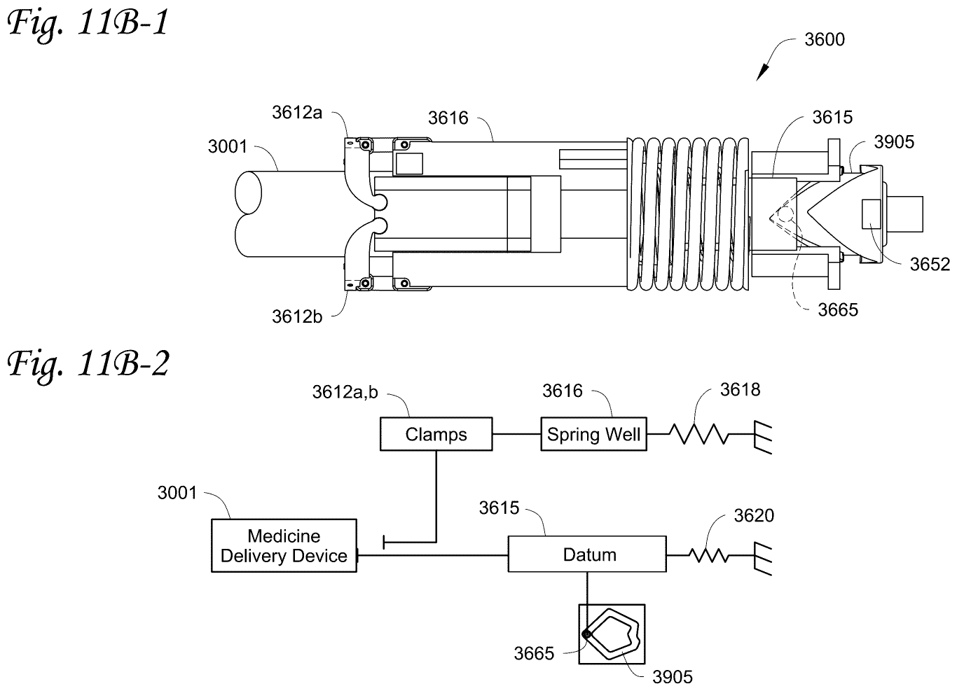

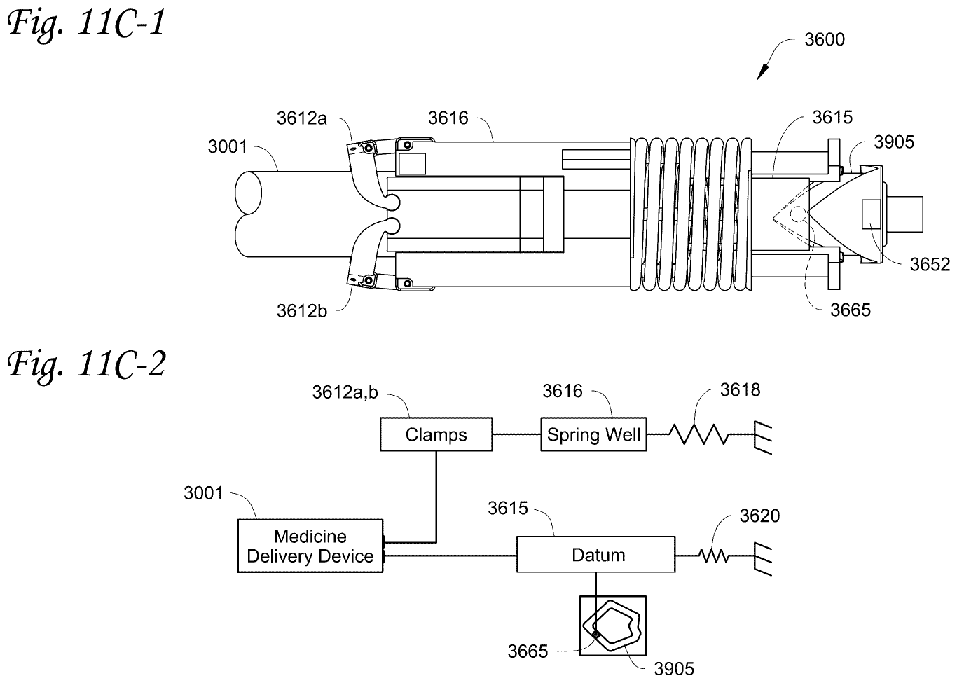

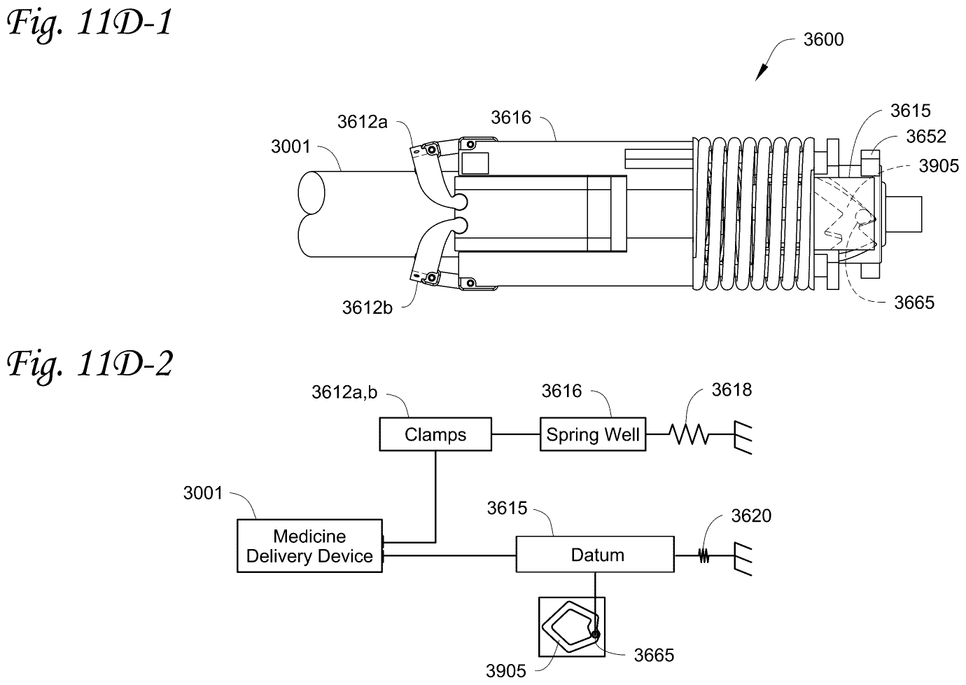

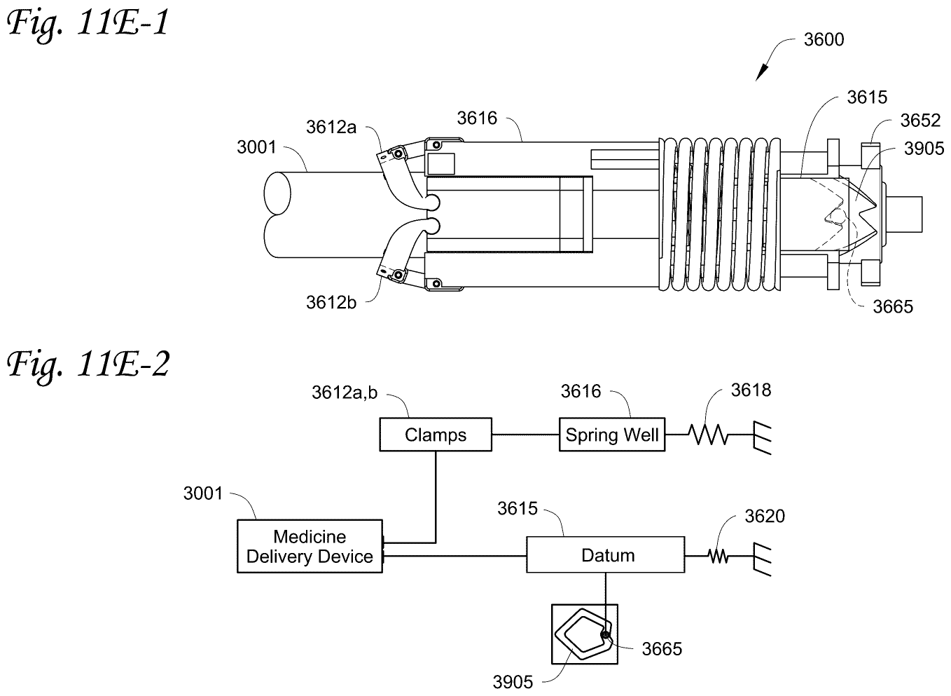

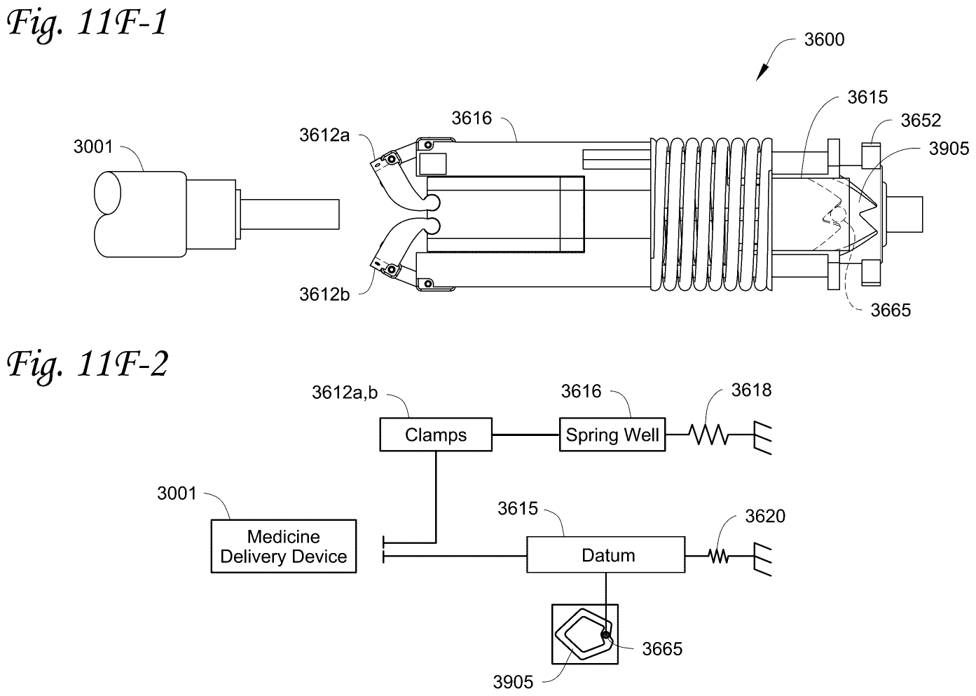

FIGS. 11A-1-11F-2 illustrates states of the clamping mechanism 3600 of the reusable accessory 3000 when receiving the medication delivery device 3001 in the housing 3100. FIGS. 11A-1, 11B-1, 11C-1, 11D-1, 11E-1, 11F-1 each show a side view of the clamping mechanism 3600 in different states. FIGS. 11A-2, 11B-2, 11C-2, 11D-2, 11E-2, 11F-2 each show a schematic view of the clamping mechanism 3600 in the different states.

FIGS. 11A-1 and 11A-2 show the clamping mechanism 3600 in a first state. The first state is when the medication delivery device 3001 is located outside the reusable accessory 3000 (e.g., prior to inserting the medication delivery device 3001 into the reuseable accessory 3000. In the first state, the medication delivery device 3001 is not in contact with the clamps 3612a,b and is not in contact with the datum 3615. The datum 3615 is in a first position (i.e., relative to the housing 3100). For example, the guide(s) 3665 of the datum 3615 are in a first position in the guide path 3905 in the clicker portion 3624. The first spring 3618 is in a relatively less compressed state. The second spring 3620 is in a relatively less compressed state.

FIGS. 11B-1 and 11B-2 show clamping mechanism 3600 in a second state. The second state is when the medication delivery device 3001 is inserted into the reuseable accessory 3000 and contacts the datum 3615 (e.g., first contact with the datum 3615, without moving the datum 3615). In the second state, the medication delivery device 3001 is not in contact with the clamps 3612a,b (e.g., not clamped by the clamps 3612a,b). The datum 3615 is in the first position (i.e., relative to the housing 3100). For example, the guide(s) 3665 of the datum 3615 are still in the first position in the guide path 3905 in the clicker portion 3624. The first spring 3618 is in the relatively less compressed state. The second spring 3620 is in the relatively less compressed state.

FIGS. 11C-1 and 11C-2 show clamping mechanism 3600 in a third state. The third state is when the medication delivery device 3001 further inserted into the reusable accessory 3000 and pushes on and moves the datum 3615 (e.g., moves the datum relative to the spring well 3616). The movement of the datum 3615 also moves the clamps 3612a,b into contact with the medication delivery device 3001 (e.g., clamps 3612a,b each rotate radially inward into contact with the medication delivery device 3001). In the third state, the clamps 3612a,b contact the medication delivery device 3001 (e.g., moved into contact with the medication delivery device 3001, not applying a substantial clamping force). The datum 3615 is in a second position (i.e., relative to the housing 3100). For example, the guide(s) 3665 of the datum 3615 are in a second position in the guide path 3905 in the clicker portion 3624. Each of the second position of the datum 3615 and the second position of the guide(s) is further away from the distal end 3010 of the housing 3100 along the housing axis X than the first position. The first spring 3618 is in the relatively less compressed state. The second spring 3620 is in the relatively more compressed state (e.g., in a second compressed state that is greater than a first compressed state in states 1 and 2).

FIGS. 11D-1 and 11D-2 show clamping mechanism 3600 in a fourth state. The fourth state is when the medication delivery device 3001 is further inserted into the reusable accessory 3000 and further moves the datum 3615 (e.g., moves the datum 3615 relative to the spring well 3616). The movement of the datum 3615 also causes the clamps 3612a,b to apply a clamping force on the medication delivery device 3001 (e.g., clamps 3612a,b push towards each other in contact with the medication delivery device 3001, which camps the medication delivery device 3001 between the two clamps 3612a,b). In the fourth state, the clamps 3612a,b are configured to clamp the medication delivery device 3001 in place (e.g., apply a substantial clamping force on the medication delivery device 3001). The datum 3615 is in a third position (i.e., relative to the housing 3100). For example, the guide(s) 3665 of the datum 3615 are in a third position in the guide path 3905 in the clicker portion 3624. The third position can be a maximum axial position of the datum 3615 (e.g., greatest distance away from the distal end 3010 of the housing 3100 along the housing axis X). Each of the third position of the datum 3615 and the third position of the guide(s) 3665 are further away from the distal end 3010 of the housing 3100 along the housing axis X than the first and second positions. For example, the clicker portion 3624 limits further travel of the datum 3615. The first spring 3618 is in a relatively more compressed state (e.g., in a second compressed state that is more compressed than a first compressed state in states one, two, and three). The second spring 3620 is in a (relatively) greatly compressed state (e.g., in a third compressed state that is more compressed than the first compressed state in states one and two and the second compressed state in state three).

FIGS. 11E-1 and 11E-2 show clamping mechanism 3600 in a fifth state. The fifth state occurs after a user releases the medication delivery device 3001 inserted into the reusable accessory 3000, and the datum 3615 is moved backward into a fourth position (e.g., moved towards the distal end 3010 of the housing 3100). The fifth state is a stable state (e.g., remains in the fifth state without any external force on the medication delivery device 3001) in which the clamps 3612 are configured to clamp the medication delivery device 3001 in place. In the fifth state, the clamping mechanism 3600 is reversibly attached to the medication delivery device 3001 (e.g., by the clamps 3612a,b clamping onto and holding the medication delivery device 3001). For example, in the fifth state, the clamping mechanism 3600 may be configured to prevent direct removal of the medication delivery device 3001 from the housing 3100 (e.g., in the fifth state configured to prevent the medication delivery device 3001 from being pulled directly out of the housing 3100 (e.g., pulled in the left direction in FIG. 10D-1)).

For example, the guide(s) 3665 of the datum 3615 are in a fifth position in the guide path 3905 in the clicker portion 3624. Each of the fourth position of the datum 3615 and the fourth position of the guide(s) 3665 is between the third and second positions along the housing axis X. The first spring 3618 is in the relatively more compressed state (e.g., in the second compressed state). The second spring 3620 is in a (relatively) intermediate compressed state (e.g., in a third compressed state that is more compressed than the second compressed state and is less compressed than the first compressed state).

In the fifth state, the clamping mechanism 3600 of the reuseable accessory 3000 is configured to allow removal of the medication delivery device 3001 from the housing 3100 by pushing/inserting the medication delivery device 3001 further into the housing 3100. This causes the clamping mechanism 3600 to return to the third state (e.g., shown in FIGS. 10C-1 and 10C-2) in which the clamping mechanism 3600 does not apply a clamping force on the medication delivery device 3001 (e.g., the clamps 3612a,b are just in contact with the medication delivery device 3001 without applying a substantial clamping force).

FIGS. 11F-1 and 11F-2 show the clamping mechanism 3600 in a sixth state. The sixth state occurs when a user pulls the medication delivery device 3001 out of the clamping mechanism 3600 while still clamped on the medication delivery device 3001 (e.g., pulls the medication delivery device 3001 from the clamping mechanism 3600 in the fifth state without returning the clamping mechanism 3600 to the second state). In the sixth state, the clamping mechanism 3600 is still engaged to apply clamping to a medication delivery device that is no longer disposed in the housing 3100 of the reusable accessory 3000. For example, a user may reset the clamping mechanism 3600 in a sixth state to the first state by further inserting a medication delivery device into the housing 3100 to cause the datum 3615 to move back into the second state.

For example, the guide(s) 3665 of the datum 3615 are in the fourth position in the guide path 3905 in the clicker portion 3624. The first spring 3618 is in the relatively more compressed state (e.g., in the second compressed state). The second spring 3620 is in the intermediate compressed state.

The attachment sensors 3050, 3052 of the reusable accessory 3000 include a spring well sensor 3050 and a clicker sensor 3052. The spring well sensor 3050 is disposed along a spring well 3616 of the clamping mechanism 3600. The spring well sensor 3050 is configured to detect when the spring well 3616 moves into a specific position relative to the housing 3100. For example, the spring well sensor 3050 is configured to detect when the spring well sensor 3050 moves relative to the housing 3100 into a specific position (e.g., moves into an improper removal position, e.g., in the position of the sixth state in FIGS. 11F-1 and 11F-2).

The spring well 3616 includes a position indicator 3650. The position indicator 3650 triggers the spring well sensor 3050 when moved into a position adjacent to the spring well sensor 3050. For example, the position indicator 3650 can trigger the spring well sensor 3050 when the position indicator 3650 stops in adjacent to the spring well sensor 3050 and when the position indicator 3650 continuously moves into and past being adjacent to the spring well sensor 3050.

The spring well sensor 3050 can be a proximity sensor. For example, the position indicator 3650 may be protrusion on the spring well 3616, and the spring well sensor 3050 may be an infrared sensor that detects when the protrusion is in a position adjacent to the infrared sensor. For example, the position indicator 3650 may be magnet of the spring well 3616 (e.g., disposed on or in the spring well 3616), and the spring well sensor 3050 may be a hall effect sensor that detects when the magnet is in the position adjacent to the hall effect sensor.

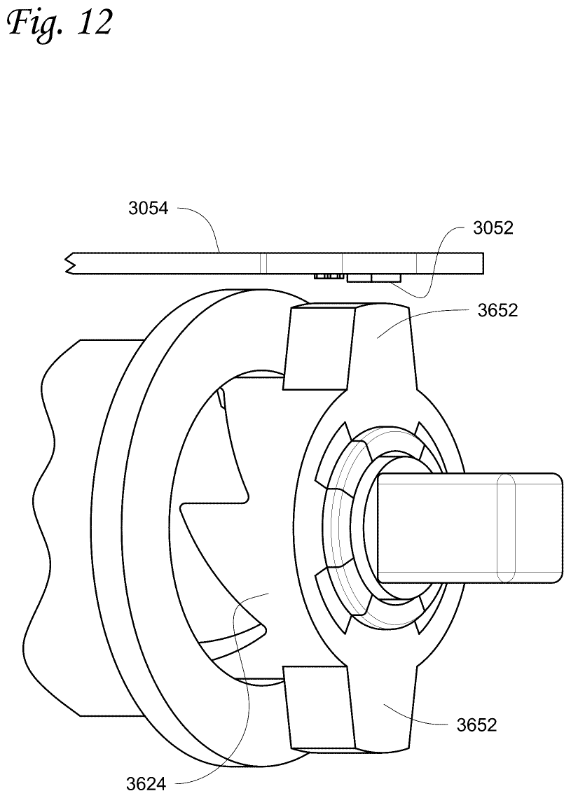

The clicker sensor 3052 is disposed along a clicker portion 3624 of the clamping mechanism 3600. FIG. 12 is a partial perspective side view of the clicker portion 3624, the clicker sensor 3052, and the circuit board 3054 of the clicker retainer 3622 of the reusable accessory 3000.

The clicker sensor 3052 is configured to detect when the clicker portion 3624 rotates relative to the housing 3100 into one or more specific position(s). The clicker sensor 3052 is configured to detect when the clicker portion 3624 is in a position that corresponds with the clamping mechanism 3600 moving into and/or being in its engaged/clamped position (e.g., clamps 3612a,b positioned to clamp onto any medication delivery device disposed therebetween, the clamping mechanism 3600 in the fifth state (e.g., clamping mechanism 3600 in the fifth state in FIGS. 11E-1 and 11E-2), the clamping mechanism 3600 in the sixth state (e.g., medication delivery device 3001 in FIGS. 11F-1 and 11F-2)). In one example, the clicker sensor 3052 detects when the clamping mechanism 3600 is in the engaged/clamped position. In another example, the clicker sensor 3052 detects when the clamping mechanism 3600 is in a position indicating that the clamping mechanism 3600 is moving into the engaged/clamped position (e.g., detects when the clamping mechanism 3600 is in the fourth state).

The clicker portion 3624 includes one or more rotation indicators 3652. The rotation indicator(s) 3652 can be disposed on an outer circumference of the clicker portion 3624. As shown in the illustrated embodiment, the rotation indicator(s) 3652 can be disposed on the second clicker body 3628 of the clicker portion 3624. In the illustrated embodiment, the guide path 3905 is formed such that the clamping mechanism 3600 is moves into the engaged/clamped position twice in every full rotation of the clicker portion 3624. For example, the moving of the clamping mechanism 3600 from the zero state to the fifth state rotates the clicker portion 3624 by 180 degrees. In the illustrated embedment, the clamping mechanism 3600 has the two engaged/clamped positions and the clicker portion 3624 correspondingly has two of the rotation indicators 3652. For example, as shown in FIG. 12, the two rotation indicators 3652 are provided at opposite sides of the clicker portion 3624. It should be appreciated that the guide path 3905 may be formed to provide a different number of engaged/clamped positions in a full rotation of the clicker portion 3624, with the clicker portion 3624 having a correspondingly different number of rotation indicators 3652.

Each rotation indicator 3652 triggers the clicker sensor 3052 when moved (e.g., rotated) into a position adjacent to the clicker sensor 3052. For example, a rotation indicator 3652 can trigger the clicker sensor 3052 when a rotation indicator 3650 is rotated into a position that is adjacent to the clicker sensor 3052. The rotation of the clicker portion 3624 recurringly rotates the clicker sensor 3052 into the position adjacent to the clicker sensor 3052. For example, FIG. 12 shows the clicker portion 3624 in a rotational position in which one of the rotation indicator 3652 (an upper rotation indicator 3650 on the page in FIG. 12) is adjacent to the clicker sensor 3052.

The clicker sensor 3052 can be a proximity sensor. For example, each position indicator 3652 can be protrusion of the clicker portion 3624 (e.g., a protrusion extending radially outward from the clicker portion 3624, from one of the clicker bodies 3626, 3628 of the clicker portion 3624), and the clicker sensor 3052 may be an infrared sensor that detects when the protrusion when in the position adjacent to the infrared sensor. For example, the position indicator 3652 may be magnet of the clicker portion 3052 (e.g., disposed on and/or in the spring well clicker portion 3052, disposed in and/or on the protrusion the clicker portion 3624), and the clicker sensor 3052 may be a hall effect sensor that detects when the magnet is rotated to be in the position adjacent to the hall effect sensor.

As previously discussed, the attachment sensors 3050, 3052 are configured to detect the reversible attachment of the adaptive element (e.g., the clamping mechanism 3600) to a medication delivery device. For example, the sensing from the clicker sensor 3052 can indicate whether the clamping mechanism 3600 is in the engaged/clamped position, and the sensing from the spring well sensor 3050 can indicate whether the clamping mechanism 3600 is in the fifth state (e.g., medication delivery device 3001 clamped in the clamping mechanism 3600) or in the sixth state (e.g., the clamping mechanism 3600 clamping but without a medication delivery device 3001 present, the medication delivery device 3001 was improperly removed). Accordingly, the attachment sensors 3050, 3052 in the illustrated embodiment are used to detect whether the adaptive element (e.g., clamping mechanism 3600) is reversibly attached (e.g., clamped onto) a medication delivery device in the housing 3100. For example, the controller of the reuseable accessory 3000 (e.g., printed circuit board 3054) receives sensing data from each of the attachment sensors 3050, 3052 (e.g., sensing data from the clicker sensor 3052 and sensing data from the spring well sensor 3050), and the controller is configured to detect the reversible attachment of the adaptive element (e.g., clamping mechanism 3600) to a medication delivery device based on the received sensing data.

### ASPECTS

Any of Aspects 1-22 may be combined with any of Aspects 23-34, any of Aspects 23-29 any be combined with any of Aspects 30-34, and any of Aspects 30-33 may be combined with Aspect 34.
Aspect 1. A reusable accessory for receiving a medication delivery device, comprising:
   a housing configured to receive a medication delivery device;
   an adaptive element coupled to the housing, the adaptive element being configured to reversibly attach to the medication delivery device; and
   one or more attachment sensors coupled to the housing, the one or more attachment sensors configured to detect the adaptive element reversibly attaching to the medication delivery device.
Aspect 2. The reusable accessory of Aspect 1, wherein the one or more attachment sensors include an accelerometer configured to detect the one or more adaptive elements reversibly attaching to the medication delivery device.
Aspect 3. The reusable accessory of Aspect 2, wherein the one or more attachment sensors include:
   one or more proximity sensors coupled to the housing, the one or more proximity sensors being configured to detect the medication delivery device being inserted into the reusable accessory.
Aspect 4. The reusable accessory of Aspect 3, wherein the accelerometer changes from a standby mode to a high sensitivity mode upon the detecting of the medication delivery device being inserted into the reusable accessory.
Aspect 5. The reusable accessory of any one of Aspects 3-4, wherein the one or more proximity sensors includes a first proximity sensor positioned on a distal end of the reusable accessory, and a second proximity sensor positioned on the proximal end of the reusable accessory.
Aspect 6. The reusable accessory of Aspect 5, wherein the one or more proximity sensors includes a third proximity sensor positioned between the first and second proximity sensors.
Aspect 7. The reusable accessory of any one of Aspects 4-6, wherein the one or more proximity sensors include an optical proximity sensor.
Aspect 8. The reusable accessory of any one of Aspects 4-7, wherein the one or more proximity sensors include a pressure sensor.
Aspect 9. The reusable accessory of any one of Aspects 3-8, wherein the accelerometer is configured to provide a haptic feedback in response to the adaptable element reversibly attaching to the medication delivery device.
Aspect 10. The reusable accessory of any one of Aspects 3-8, wherein the accelerometer is configured to detect the reusable accessory being at least one of picked up or put down.
Aspect 11. The reusable accessory of any one of Aspects 1-10, wherein the adaptable element is an interchangeable snap ring reversibly coupled to a distal end of the housing of the reusable accessory device, wherein the interchangeable ring is configured to fit a geometry of a specific medication delivery device.
Aspect 12. The reusable accessory of any one of Aspects 1-11, wherein the adaptable element comprises an interchangeable collar reversibly coupled to the housing, wherein the interchangeable collar is configured to fit the medication delivery device.
Aspect 13. The reusable accessory of any one of Aspects 1-12, further comprising:
   at least one user interface configured to communicate one or more notifications including an alert that the medication delivery device is not fully inserted into the reusable accessory, a time that the sensor last detected the adaptive element reversibly attaching to the medication delivery device, or whether there has been a missed dose.
Aspect 14. The reusable accessory of any one of Aspects 1-13, wherein the reuseable accessory is a pen cap for an insulin pen that is the medication delivery device.
Aspect 15. The reusable accessory of any one of Aspects 1-14, wherein an adaptive element is a clamping mechanism configured to reversibly attach to the medication delivery device by clamping onto the medication delivery device received in the housing.
Aspect 16. The reusable accessory of Aspect 15, wherein the clamping mechanism has a plurality of states, and the one or more attachment sensors are configured to detect a current state of the clamping mechanism from the plurality of states.
Aspect 17. The reusable accessory of any one of Aspects 15-16, wherein the clamping mechanism includes a clicking portion configured to rotate relative to the housing, and the one or more attachment sensors including a clicker sensor disposed adjacent to the clicking portion and to configured to detect when the clicking portion is in at least one rotational position.
Aspect 18. The reusable accessory of Aspect 17, wherein the clicking portion includes one or more rotational indicators, the rotation of the clicker portion causes the rotational indicator to recurringly rotate into a position adjacent to the clicking sensor in which the clicking sensor detects the rotational indicator.
Aspect 19. The reusable accessory of any one of Aspects 17-18, wherein clicking portion includes one or more rotational indicators, and the one or more rotational indicators are rotated into a position adjacent to the clicking sensor when the clamping mechanism is in a clamped position configured to apply a clamping force to the medication delivery device.
Aspect 20. The reusable accessory of any one of Aspects 15-19, wherein the one or more attachment sensors include a spring well sensor configured to detect for a spring well of the clamping mechanism being in a specific position relative to the housing.
Aspect 21. The reusable accessory of Aspect 20, wherein a spring well includes a position indicator, and the spring well sensor detects spring well being in the specific position by detecting the position indicator moving into a position adjacent to the spring well sensor.
Aspect 22. The reusable accessory of any one of Aspects 15-21, wherein the one or more attachment sensors configured to detect an improper removal of the medication delivery device caused by removal of medication delivery device while still being clamping onto by the clamping mechanism.
Aspect 23. A method for detecting a medication delivery device in a reusable accessory, the reusable accessory comprising a housing configured to receive the medication delivery device, an adaptable element coupled to the housing, and one or more attachment sensors, the method comprising:
   receiving, via the one or more attachment sensors, sensing data for receiving the medication delivery device in the housing of the reusable accessory;
   determining, based on the received sensing data, whether the medication delivery device is reversibly attached, via the adaptable element, to the housing of the reusable accessory; and presenting, via the at least one user interface, a status of the medication delivery device being reversibly attached to the reusable accessory via the adaptable element.
Aspect 24. The method of Aspect 23, further comprising:
   receiving, via one or more proximity sensors of the one or more attachment sensors, sensing data to detect insertion of the medication delivery device into the housing of the reusable accessory.
Aspect 25. The method of Aspect 24, wherein the detecting of the medication delivery device being inserted into the housing comprises:
   detecting, via a first one of the one or more proximity sensors adjacent to an open end of the housing, the medication delivery device entering a first position within the housing.
Aspect 26. The method of Aspect 25, wherein the detecting of the medication delivery device being inserted into the housing comprises:
   detecting, via a second one of the proximity sensors adjacent to a closed end of the housing, the medication delivery device entering a second position of the housing.
Aspect 27. The method of Aspect 26, wherein the one or more attachment sensors include an accelerometer, the accelerometer detects a snap-fit attachment between the adaptable element and the medication delivery device upon the detecting of the medication delivery device entering the second position of the housing.
Aspect 28. The method of any one of Aspects 25-27, further comprising:
   changing an accelerometer of the one or more attachment sensors from a standby mode to a high sensitivity mode upon the detecting of the medication delivery device entering the first position within the housing.
Aspect 29. The method of any one of Aspects 23-28, further comprising:
   the one or more attachment sensors include an accelerometer configured to detect for an acceleration corresponding to the reversibly attachment of the adaptable element to the medication delivery device.
Aspect 30. A reusable accessory adaption kit comprising:
   a first reuseable accessory including a housing and one or more attachment sensors coupled to the housing;
   a first adaptive element configured to reversibly attach to a first medication delivery device while reversibly coupled to the housing of the reusable accessory; and
   a second adaptive element different from the first adaptive element, the second adaptive element configured to reversibly attach to a second medication delivery device while reversibly coupled to the housing of the reusable accessory, a geometry of the first medication delivery device different from a geometry of the second medication delivery device,
   wherein the one or more attachment sensors are configured to detect:
      the reversible attachment of the first adaptive element to the first medication delivery device, when the first medication delivery device is received in the housing of the reuseable accessory, and
      the reversible attachment of the second adaptive element to the second medication delivery device, when the second medication delivery device is received in the housing of the reuseable accessory.
Aspect 31. The reusable accessory adaption kit of Aspect 30, wherein the first adaptive element is a first interchangeable collar, and the second adaptive element is a second interchangeable collar.
Aspect 32. The reusable accessory adaption kit of Aspect 30, wherein the first adaptive element is a first snap ring and the second adaptive element is a second snap ring.
Aspect 33. The reusable accessory adaption kit of any one of Aspects 30-32, wherein
   the geometry of the second medication delivery device is incompatible with revisable attachment via the first adaptive element, and
   the geometry of the first medication delivery device is incompatible with the revisable attachment via the second adaptive element.
Aspect 34. An adaptable reusable accessory kit comprising:
   a reuseable accessory including a housing and one or more attachment sensors coupled to the housing; and
   a plurality of adaptive elements for a plurality of delivery medication devices with different geometries, the plurality of adaptive elements each configured to reversibly attach to a corresponding medication delivery device in the plurality of delivery medication devices while reversibly coupled to the housing of the reusable accessory,
   wherein the one or more attachment sensors are configured to detect, for each adaptive element in the plurality of adaptive elements when reversibly coupled to the housing, the reversible attaching of said adaptive element to the corresponding medication delivery device received in the housing.

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

It is to be understood that different features, variations, and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

What is claimed is:

1. A reusable accessory for receiving a medication delivery device, comprising:
a housing configured to receive a medication delivery device;
an adaptive element coupled to the housing, the adaptive element being configured to reversibly attach to the medication delivery device; and
one or more attachment sensors coupled to the housing, the one or more attachment sensors configured to detect the adaptive element reversibly attaching to the medication delivery device.

2. The reusable accessory of claim 1, wherein the one or more attachment sensors include an accelerometer configured to detect the one or more adaptive elements reversibly attaching to the medication delivery device.

3. The reusable accessory of claim 2, wherein the one or more attachment sensors include:
one or more proximity sensors coupled to the housing, the one or more proximity sensors being configured to detect the medication delivery device being inserted into the reusable accessory, and
the accelerometer changes from a standby mode to a high sensitivity mode upon the detecting of the medication delivery device being inserted into the reusable accessory.

4. The reusable accessory of claim 1, wherein the adaptable element is an interchangeable snap ring reversibly coupled to a distal end of the housing of the reusable accessory device, wherein the interchangeable ring is configured to fit a geometry of a specific medication delivery device.

5. The reusable accessory of claim 1, wherein the adaptable element comprises an interchangeable collar reversibly coupled to the housing, wherein the interchangeable collar is configured to fit the medication delivery device.

6. The reusable accessory of claim 1, further comprising:
at least one user interface configured to communicate one or more notifications including an alert that the medication delivery device is not fully inserted into the reusable accessory, a time that the sensor last detected the adaptive element reversibly attaching to the medication delivery device, or whether there has been a missed dose.

7. The reusable accessory of claim 1, wherein the reuseable accessory is a pen cap for an insulin pen that is the medication delivery device.

8. The reusable accessory of claim 1, wherein an adaptive element is a clamping mechanism configured to reversibly attach to the medication delivery device by clamping onto the medication delivery device received in the housing.

9. The reusable accessory of claim 8, wherein the clamping mechanism has a plurality of states, and the one or more attachment sensors are configured to detect a current state of the clamping mechanism from the plurality of states.

10. The reusable accessory of claim 8, wherein the clamping mechanism includes a clicking portion configured to rotate relative to the housing, and the one or more attachment sensors including a clicker sensor disposed adjacent to the clicking portion and to configured to detect when the clicking portion is in at least one rotational position.

11. The reusable accessory of claim 10, wherein the clicking portion includes one or more rotational indicators, the rotation of the clicker portion causes the rotational indicator to recurringly rotate into a position adjacent to the clicking sensor in which the clicking sensor detects the rotational indicator.

12. The reusable accessory of claim 10, wherein clicking portion includes one or more rotational indicators, and the one or more rotational indicators are rotated into a position adjacent to the clicking sensor when the clamping mechanism is in a clamped position configured to apply a clamping force to the medication delivery device.

13. The reusable accessory of claim 8, wherein the one or more attachment sensors include a spring well sensor configured to detect for a spring well of the clamping mechanism being in a specific position relative to the housing.

14. The reusable accessory of claim 13, wherein a spring well includes a position indicator, and the spring well sensor detects spring well being in the specific position by detecting the position indicator moving into a position adjacent to the spring well sensor.

15. The reusable accessory of claim 8, wherein the one or more attachment sensors configured to detect an improper removal of the medication delivery device caused by removal of medication delivery device while still being clamping onto by the clamping mechanism.

16. A method for detecting a medication delivery device in a reusable accessory, the reusable accessory comprising a housing configured to receive the medication delivery device, an adaptable element coupled to the housing, and one or more attachment sensors, the method comprising:
receiving, via the one or more attachment sensors, sensing data for receiving the medication delivery device in the housing of the reusable accessory;
determining, based on the received sensing data, whether the medication delivery device is reversibly attached, via the adaptable element, to the housing of the reusable accessory; and
presenting, via the at least one user interface, a status of the medication delivery device being reversibly attached to the reusable accessory via the adaptable element.

17. The method of claim 16, wherein the detecting of the medication delivery device being inserted into the housing comprises:
detecting, via a first proximity sensor of the one or more attachment sensors disposed adjacent to an open end of the housing, the medication delivery device entering a first position within the housing; and
changing at least one of the one or more attachment sensors from a standby mode to a high sensitivity mode upon the detecting of the medication delivery device entering the first position within the housing.

18. The method of claim 16, further comprising:
the one or more attachment sensors include an accelerometer configured to detect for an acceleration corresponding to the reversibly attachment of the adaptable element to the medication delivery device.

19. A reusable accessory adaption kit comprising:
a first reuseable accessory including a housing and one or more attachment sensors coupled to the housing;
a first adaptive element configured to reversibly attach to a first medication delivery device while reversibly coupled to the housing of the reusable accessory; and
a second adaptive element different from the first adaptive element, the second adaptive element configured to reversibly attach to a second medication delivery device while reversibly coupled to the housing of the reusable accessory, a geometry of the first medication delivery device different from a geometry of the second medication delivery device,
wherein the one or more attachment sensors are configured to detect:
the reversible attachment of the first adaptive element to the first medication delivery device, when the first medication delivery device is received in the housing of the reuseable accessory, and
the reversible attachment of the second adaptive element to the second medication delivery device, when the second medication delivery device is received in the housing of the reuseable accessory.

20. The reusable accessory adaption kit of claim 19, wherein
the geometry of the second medication delivery device is incompatible with revisable attachment via the first adaptive element, and
the geometry of the first medication delivery device is incompatible with the revisable attachment via the second adaptive element.
